SEQUENCE LISTING (26 sequences)

SEQ ID NO: 1 - protein, 23 aa, Homo sapiens
IGAQVYEQVLRSAYAKRNSSVND

SEQ ID NO: 2 - protein, 19 aa, Homo sapiens
IGMHMIRLYTNKNSTLNGT

SEQ ID NO: 3 - protein, 5 aa, Homo sapiens
PERLP

SEQ ID NO: 4 - protein, 11 aa, Homo sapiens
PTIKLGGHWKP

SEQ ID NO: 5 - protein, 10 aa, Homo sapiens
PRWKVAILIP

SEQ ID NO: 6 - protein, 10 aa, Homo sapiens
PFRNRHEHLP

SEQ ID NO: 7 - protein, 9 aa, Homo sapiens
PVLFRHLLP

SEQ ID NO: 8 - protein, 12 aa, Homo sapiens
PEGDTGKYKSIP

SEQ ID NO: 9 - protein, 8 aa, Homo sapiens
PENFTYSP

SEQ ID NO: 10 - protein, 4 aa, Homo sapiens
PYLP

SEQ ID NO: 11 - protein, 7 aa, Homo sapiens
PCPEKLP

SEQ ID NO: 12 - protein, 7 aa, Homo sapiens
PGGHWRP

SEQ ID NO: 13 - protein, 10 aa, Homo sapiens
PRWKVAVLIP

SEQ ID NO: 14 - protein, 9 aa, Homo sapiens
PIFFLHLIP

SEQ ID NO: 15 - protein, 12 aa, Homo sapiens
PEGDLGKYKSIP

SEQ ID NO: 16 - protein, 5 aa, Homo sapiens
PELAP

SEQ ID NO: 17 - DNA, 21 bp, Artificial Sequence
  Description of Combined DNA/RNA Molecule Synthetic oligonucleotide
cggagugaguggcuuaacatt

SEQ ID NO: 18 - DNA, 21 bp, Artificial Sequence
  Description of Combined DNA/RNA Molecule Synthetic oligonucleotide
uguuaagccacucacuccgtt

SEQ ID NO: 19 - protein, 8 aa, Artificial Sequence
  Description of Artificial Sequence Synthetic peptide
CCPXHXXC

SEQ ID NO: 20 - DNA, 18 bp, Artificial Sequence
  Description of Artificial Sequence Synthetic primer
tgacccttctgctctgtt

SEQ ID NO: 21 - DNA, 20 bp, Artificial Sequence
  Description of Artificial Sequence Synthetic primer
tgagaggtggtgctgacatc

SEQ ID NO: 22 - DNA, 22 bp, Artificial Sequence
  Description of Artificial Sequence Synthetic primer
aggtcatcactattggcaacga

SEQ ID NO: 23 - DNA, 27 bp, Artificial Sequence
  Description of Artificial Sequence Synthetic primer
cacttcatgatggaattgaatgtagtt

SEQ ID NO: 24 - DNA, 21 bp, Artificial Sequence
  Description of Combined DNA/RNA Molecule Synthetic oligonucleotide
auggugauuagacuguacctt

SEQ ID NO: 25 - DNA, 21 bp, Artificial Sequence
  Description of Combined DNA/RNA Molecule Synthetic oligonucleotide
aagcguacuaggaucaguatt

SEQ ID NO: 26 - protein, 31 aa, Homo sapiens
VLENSTKNSNDPAVFKDNPTEDVEYQCVADN

(19) United States
(12) Patent Application Publication
(10) Pub. No.: US 2009/0202439 A1
(43) Pub. Date: Aug. 13, 2009

(54) Methods for treatment of angiogenesis

(75) Inventor: Subroto Chatterjee, Columbia, MD (US)

Correspondence Address: EDWARDS ANGELL PALMER & DODGE LLP, P.O. BOX 55874, BOSTON, MA, 02205, US

(73) Assignee: The Johns Hopkins University, Baltimore, MD (US)

(21) Appl. No.: 11/708,281

(22) Filed: Feb. 19, 2007

Related U.S. Application Data
(63) Continuation of application No. PCT/US05/29730, filed on Aug. 19, 2005.
(60) Provisional application No. 60/603,016, filed on Aug. 20, 2004.

(51) Int. Cl. A61K 39/395; A61K 31/16; A61K 31/5375; A61K 31/445; A61K 31/40; A61K 38/00; A61K 31/7105; A61K 49/00; A61P 35/00; G01N 33/53

(52) U.S. Cl. 424/9.2; 514/613; 514/231.2; 514/317; 514/408; 424/130.1; 514/2; 424/139.1; 435/4

(57) ABSTRACT

The present invention includes methods for treatment and prophylaxis of diseases, post-surgical disorders and bacterial infections associated with lactosylceramide. The methods generally provide for administration to a subject one or more compounds that alter the activity of VEGF pathway members, including LacCer synthase (GalT-V/VI), PECAM1, VEGFR, VEGF or related pathway members to treat a subject suffering from or susceptible to a condition caused or contributed to by VEGF. The present invention also relates to methods for detecting and analyzing compounds with therapeutic capacity to treat such condition.

### CROSS REFERENCE TO RELATED APPLICATIONS

This application claims priority to U.S. Provisional Application No. 60/603,016, filed Aug. 20, 2004, and entitled, “Methods for Treating Angiogenesis Modified by Vascular Endothelial Growth Factor and Lactosylceramide,” which is hereby incorporated by reference in its entirety.

### BACKGROUND

Angiogenesis, the sprouting of blood capillaries from existing ones is required during embryonic development and wound healing. Angiogenesis involves a series of steps, wherein endothelial cells degrade their basement membrane locally. Next, the endothelial cells migrate into the connective tissue stroma, proliferate and finally differentiate into capillary loops. VEGF is a mediator of angiogenesis and is of considerable interest, as it is known to augment collateral blood flow in experimental animals and in patients with limb and myocardial ischemia.³¹ In addition, VEGF induced neo-vascularization has been documented in atherosclerosis, diabetic retinopathy and tumor metastasis.³⁻⁵

Although most studies have focused on the role of VEGF in angiogenesis, little is known in regard to the mechanisms underlying this critical phenotypic change. In particular, the role of neutral glycosphingolipids is not known. Lactosylceramide is a member of the neutral glycosphingolipid family and plays a pivotal role by virtue of serving as a precursor for the biosynthesis of gangliosides such as monosialoganglioside GM3, disialoganglioside GD3 as well as globotriosylceramide and LacCer sulfate. While these glycosphingolipids have been shown to impart diverse biological functions, LacCer by its own right, has been implicated in cell proliferation, cell adhesion and cell migration; events that are collectively required for angiogenesis. Most importantly, LacCer was found to induce PECAM-1 gene/protein expression²²; a pre-requisite to initiate angiogenesis.^{10,11}

The sprouting of new capillaries from pre-existing blood vessels (angiogenesis and the differentiation of endothelial cells (vasculogenesis) are phenotypic events in health and diseases.¹ Vascular endothelial growth factor (VEGF) has been implicated in the process of vasculogenesis and angiogenesis.² Aberrant expression of VEGF has been reported in several vascular pathologies such as inflammation, complications of diabetes mellitus, cardiovascular diseases and tumor metastasis.³. VEGF binds to its receptors KDR/Flk-1, to mediate its effect on angiogenesis in physiological conditions and in human atherosclerosis.⁴⁻⁶

Platelet endothelial cell adhesion molecule (PECAM-1)/CD31 is a constitutively expressed integral protein in endothelial cells.⁷ In addition, PECAM-1 is expressed in platelets, monocytes, neutrophils and a certain subset of T cells.⁸ Recent studies implicate a potential role of PECAM-1 in angiogenesis and in vitro endothelial cell migration.^{9,10} For example, implantation of a human mesoendothelioma cell line (REN), deficient in PECAM-1, failed to induce angiogenesis in nude mice. In contrast, REN cells¹⁰, over expressing PECAM-1, did induce angiogenesis in these mice.¹¹ Furthermore, the use of monoclonal PECAM-1 antibody inhibited tumor angiogenesis in mice.¹² More recently, the pivotal role of PECAM-1 in angiogenesis was unraveled, in the study reported by O'Brein et al.¹³ They observed that transfection of human full length PECAM-1 cDNA carrying mutation in immuno-tyrosine based inhibitory motifs (ITIM) in REN cells, inhibited migration of these cells in response to VEGF as well as failed to form tubes in the in vitro angiogenesis assays.

Lactosylceramide (LacCer) is a member of the glycosphingolipid (GSL) family. LacCer is ubiquitously present in mammalian tissues and plays a pivotal role as a precursor for the synthesis of complex GSLs.¹⁴ Moreover, LacCer has been implicated in critical phenotypic changes such as proliferation and adhesion in mammalian cells.¹⁵⁻²¹ Recently, in a pro-monocytic cell line (U-937), we have shown that LacCer stimulates the transcriptional expression and protein expression of PECAM-1 by recruiting PKC α and ε and PLA₂.²² Increased level of LacCer has been reported in plasma of patients with familial hypercholesterolemia²³ and in calcified and uncalcified plaques in the artery of patients who died of myocardial infraction.^{24,25} Similarly, increased plasma level of soluble PECAM-1 has been reported in patients with cardiovascular disease²⁶ and in animal models of atherosclerosis such as the apoE knockout mice.²⁷

Since PECAM-1 expression may be a pre-requisite for VEGF induced vasculogenesis and also angiogenesis, and since LacCer can up-regulate PECAM-1 expression in U937 cells, we rationalized that LacCer may well play a second messenger role in VEGF induced PECAM-1 expression and angiogenesis in human endothelial cells. In this application we disclose that LacCer is critical to mediate VEGF induced PECAM-1 expression and angiogenesis in HUVECs.

Consequently, there is a need in the art to find diagnostic methods, treatments and method of screening for new treatments for angiogenesis. Thus, it would be desirable to have additional methods of treating conditions or diseases modulated by lactosylceramides, e.g. to inhibit GalT-V, VEGFR, VEGF, PECAM-1 or other pathway members, to treat or prevent angiogenesis.

### SUMMARY OF THE INVENTION

The present invention includes methods for treatment and prophylaxis of diseases associated with lactosylceramide (LacCer). In particular, we have discovered therapies that include altering activity of one or more of LacCer synthase (GalT-V), PECAM1, VEGFR or related pathway members to treat a subject suffering from or susceptible to a disease or condition involving angiogenesis caused and/or contributed to by lactosylceramide. The present invention also relates to methods for detecting and analyzing compounds with therapeutic capacity to treat such conditions.

More specifically, the invention provides methods for treatment of proliferative disorders involving angiogenesis and related to angiogenesis, e.g. cancer, coronary heart disease, tumor metastasis, inflammatory vascular disease, inflammation, ischemia-reperfusion injury, hypertension or diabetes. In addition, the invention provides method for treatment of disorders related to tissue degradation related to angiogenesis, including, for example, intrauterine growth of a fetus, systemic sclerosis, wound healing, ischemia, reperfusion injury, diabetes, coronary artery disease, tumor growth.

Provided herein according to one aspect are methods for treating a subject suffering from or susceptible to angiogenesis comprising administering to the subject a therapeutically effective amount of a vascular endothelial growth factor (VEGF) pathway inhibitor.

In one embodiment, the angiogenesis is related to cancer, coronary heart disease, tumor metastasis, inflammatory vascular disease, or diabetes.

In another embodiment, the VEGF pathway comprises the interaction or involvement of one or more of lactosylceramide synthase (LacCer synthase, e.g., GalT-V/VI), vascular endothelial growth factor (VEGF), vascular endothelial growth factor receptor (VEGFR), platelet endothelial cell adhesion molecule 1 (PECAM-1), phospholipase A2 (PLA2) and lactosylceramide (LacCer).

According to one embodiment, methods may further comprise identifying the subject in need of treatment for angiogenesis. In a related embodiment, the identification of the subject in need of treatment compromises diagnosis of cancer, coronary heart disease, tumor metastasis, inflammatory vascular disease, ischemia-reperfusion injury, hypertension, or diabetes.

In another embodiment, a VEGF inhibitor and/or activator is administered by being coated onto an implantable medical device, for example, a biodegradable biopolymer stent. In another embodiment, a VEGF inhibitor and/or activator is administered via stent or catheter.

According to another aspect, methods for determining the therapeutic capacity of a VEGF pathway inhibitor or activator (e.g., modulator) to reduce angiogenesis in a subject are providing and comprise performing an invasive surgical procedure on the subject; administering a VEGF pathway inhibitor to the subject; and examining the subject for vessel growth.

In one embodiment, an animal model such as tumor xenograft is used to determine the therapeutic capacity of VEGF pathway inhibitors or activators.

In another aspect, method for determining the therapeutic capacity of a candidate VEGF pathway inhibitor for treating diseases or conditions involving angiogenesis are presented and comprise providing a population of cells; contacting the cells with a candidate composition, and determining effect of the candidate composition on one or more of PECAM-1 expression, GatT-V expression, tube formation (e.g., in vitro angiogenesis assay), or LacCer level.

Methods may further comprise, according to one embodiment, contacting the cells with VEGF prior to contacting the cells with the candidate compound.

According to another aspect, are methods for treating a subject suffering from or susceptible to tissue degeneration comprising administering to the subject a therapeutically effective amount of a vascular endothelial growth factor (VEGF) pathway activator.

In one embodiment, the tissue degeneration is related to intrauterine growth of a fetus, systemic sclerosis, wound healing, ischemia, reperfusion injury, diabetes, coronary artery disease, tumor growth.

In one aspect, methods for determining the therapeutic capacity of a VEGF pathway activator to reduce tissue degeneration in a subject are provided and comprise determining pre-treatment levels of tissue degeneration in a subject;

administering a therapeutically effective amount of a VEGF pathway activator to the subject; and determining a post-treatment level of tissue degeneration in the subject.

According to one aspect, a decrease in the tissue degeneration indicates that the VEGF pathway activator is efficacious. According to a related aspect, the pre-treatment and post-treatment levels of tissue degeneration are determined in a diseased tissue.

In another related embodiment, the diseased tissue is one or more of a fetus, lung, heart, liver, vasculature (for example, cardiac ventricular microvessel formation to increase collateral blood flow to the heart or other tissue) or nervous tissue.

In one embodiment, the level of tissue degeneration is determined by PECAM-1 expression, GalT-V expression, tube formation, or LacCer level.

In another aspect, provided are methods for determining the therapeutic capacity of a candidate VEGF pathway activator for treating tissue degeneration, comprising providing a population of cells; contacting the cells with a candidate composition, and determining effect of the candidate composition on one or more of PECAM-1 expression, GalT-V expression, tube formation, or LacCer level, wherein an increase in one or more of PECAM-1 expression, GalT-V expression, tube formation, or LacCer level indicates that the candidate composition may be efficacious.

In one aspect, methods for the combination administration of VEGF pathway inhibitors and activators to treat angiogenesis related disorders. In one embodiment, biodegradable biopolymers may coated with the combination of inhibitors and activators to administer them in a time-dependant manner. In another embodiment, the inhibitors and/or activators may be coated on nanoparticles for use in single therapy or in combination therapy.

Therapies of the invention are particularly effective for the treatment and prevention of undesired angiogenesis. See the results set forth in the examples which follow.

Therapeutic methods of the invention in general comprise administering to a subject, particularly a mammal such as a primate, especially a human, a therapeutically effective amount of a compound that can alter the activity of, e.g., inhibit LacCer synthase (GalT-V/VI), PECAM1, VEGFR, VEGF, PLA2 or related pathway members to treat a subject suffering from or susceptible to a condition caused or contributed to by angiogenesis. Preferably, an administered compound inhibits angiogenesis by at least about 15% or 25% in a standard in vitro cell proliferation assay. Examples of such an assay are described below. It is generally preferred that the administered compound exhibits an IC₅₀ of at least about 500 μM in a standard in vitro VEGF pathway assay as defined below, more preferably an IC₅₀ of about 100 μM or less, still more preferably an IC₅₀ of about 1-10 μM or less in a standard in vitro VEGF pathway assay as defined below. Such compounds that can inhibit GalT-V activity are generally referred to herein as “VEGF pathway inhibitors” or other similar term.

Compounds suitable for use in the treatment methods of the invention for inhibition of angiogenesis include those of the following Formula I:

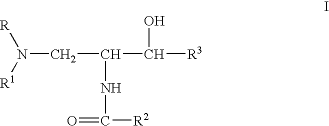

wherein R and R¹ are independently selected from the group consisting of hydrogen and straight-chained or branched C₁-C₆ alkyl with or without a substituent, and further wherein R and R¹ may be joined to form a 5, 6 or 7-membered ring;

R² is selected from the group consisting of branched or straight-chained C₆-C₃₀ alkyl with or without one to three double bonds; and

R³ is selected from the group consisting of straight-chained or branched C₆-C₂₀ alkyl with or without one to three double bonds and aryl or substituted aryl where the substituent is halo, C₁-C₄ alkoxy, methylenedioxy, C₁-C₄ mercapto, amino or substituted amino in which the amino substituent may be C₁-C₄ alkyl, or a pharmaceutically acceptable salt thereof.

In certain embodiments, R and R¹ are joined to form a 5, 6 or 7-membered ring. In related embodiments, R and R¹ are joined to form a pyrrolidino, morpholino, thiomorpholino, piperidino or azacycloheptyl ring.

Specifically preferred inhibitor compounds for use in the therapeutic methods of the invention one or more of 1-phenyl-2-decanoylamino-3-morpholino-1-propanol;
1-phenyl-2-hexadecanoylamino-3-morpholino-1-propanol;
1-phenyl-2-hexadecanoylamino-3-piperidino-1-propanol;
1-phenyl-2-hexadecanoylamino-3-pyrrolidino-1-propanol;
1-morpholino-2-hexadecanoylamino-3-hydroxyoctadec-4,5-ene;
1-pyrrolidino-2-hexadecanoylamino-3-hydroxyoctadec-4,5-ene; (1R,2R)-1-phenyl-2-decanoylamino-3-morpholino-1-propanol (D-PDMP); or trans-(2R,3R)-1-pyrrolidino-2-hexadecanoylamino-3-hydroxyoctadec-4,5-ene, chelerythrinbe chloride.

Especially preferred inhibitor compounds for use in the methods of the invention are (1R,2R)-1-phenyl-2-decanoylamino-3-morpholino-1-propanol (1) —PDMP), trans-(2R,3R)-1-pyrrolidino-2-hexadecanoylamino-3-hydroxyoctadec-4,5-ene, chelerythrinbe chloride, Gö6976, Gö6850, bromophenacyl bromide (BMB), methyl-arachidonyl fluorophosphonate (MAFP), pyrrolidine carbodithioicacid, diphenylene iodonium chloride and N-acetyl-L-cysteine.

Other suitable inhibitors include PECAM-1, LacCer, or VEGF pathway antibodies. Also included are VEGF pathway peptides and RNAi molecules.

Compounds suitable for use in the treatment methods of the invention for activation of angiogenesis include the L isomers of the following Formula I:

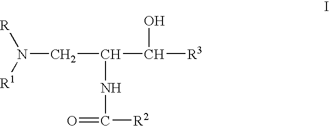

wherein R, R¹, R², and R³, are defined as described above.

Other suitable VEGF pathway inhibitor or activator compounds can be readily identified by simple testing, e.g. by in vitro testing of a candidate inhibitor compound relative to a control for the ability to inhibit or activate the VEGF pathway activity, e.g. inhibit or activate at least one pathway members' activity by at least 10% more than a control.

The invention further relates to methods of detecting and analyzing compounds that inhibit or activate VEGF pathway and exhibit therapeutic capacity to treat or prevent the above-described conditions. Preferred detection and analysis methods include both in vitro and in vivo assays to determine the therapeutic capacity of agents to modulate VEGF-responsive cells.

Preferred in vitro detection assays according to the present invention involve one or more steps associated with VEGF-related pathways. Such assays include the following steps 1) through 4):

1) culturing a population of VEGF-responsive cells with VEGF;

2) adding a known or candidate VEGF pathway inhibitor to the cells;

3) measuring activity of a specified cell molecule in the VEGF-related step; and

4) determining the effect of the known or candidate VEGF pathway inhibitor on the cell, such as cell proliferation, adhesion, expression of one or more of VEGF pathway member proteins, or tube formation.

That assay can effectively measure the capacity of the VEGF pathway inhibitor or activator to decrease or increase, respectively, VEGF pathway activity. References herein to a “standard in vitro VEGF pathway assay” or other similar phrase refers to the above protocol of steps 1) through 4) when the specified cell molecule measured in step 3) above is VEGF pathway. As described in more detail below, other in vitro assays of the invention measure additional specified cell molecules in the VEGF-related steps or pathways. The in vitro assays of the present invention can be conducted with nearly any population of cells responsive to LacCer including a lysate of such cells or tissue, or a substantially purified fraction of the lysate. Suitable LacCer responsive cells that may be employed in the assay include, e.g., cells associated with vascular intima, particularly primary and/or immortalized endothelial and smooth muscle cells, as well as certain immune cells such as leukocytes. Preferred LacCer lysates or subcellular fractions include VEGF pathway.

The in vitro detection assays of the invention can be adapted in accordance with intended use. For example, as noted above, it has been found that VEGF manifests changes in certain gene and protein expression levels and cell functions. Thus, the standard in vitro assay above can be modified at step 3) to include measuring cell proliferation or adhesion in response to the added VEGF, and to determine any effect of the VEGF pathway inhibitor on the cell function. The known or candidate VEGF pathway inhibitor tested in the assays can be employed as a sole active agent or in combination with other agents including other VEGF pathway inhibitors to be tested. In most instances, the in vitro assays are performed with a suitable control assay usually comprising the same test conditions as in the steps above, but without adding the VEGF pathway inhibitor to the medium. In such cases, a candidate VEGF pathway inhibitor can be identified as exhibiting desired activity by exhibiting at least about 10 percent greater activity relative to the control; more preferably at least about 20% greater activity relative to the control assay; and still more preferably at least about 30%, 40%, 50%, 60%, 70, 80%, 100%, 150% or 200% greater activity relative to the control. An activator will have similar activity on activation.

The invention also provides assays to detect a VEGF-responsive cell which cells may be used, e.g., in an assay of the invention as described above. For example, a potentially VEGF-responsive cell can be contacted by LacCer and then a desired cell molecule or function in a VEGF-related protein as discussed previously is measured as a function of the amount of LacCer added. In most cases, the cell is deemed responsive to LacCer if the assay employed shows at least about 10%, preferably at least about 20%, more preferably at least about 50%, and still more preferably at least about 75% or 100% change in the activity (relative to a control) of the molecule or cell function as determined by the assays provided herein. The assays can be used to identify VEGF-responsiveness in a variety of cells or tissues, including cultured cells (i.e., primary cells or immortalized cell lines) and organs.

The invention also provides in vivo assays to determine the therapeutic capacity of a known or candidate VEGF pathway inhibitor to modulate cell functions impacted by VEGF, e.g. cell proliferation and adhesion and gene and protein expression levels of VEGF pathway members. The monitored cell function suitably may be pre-existing in the test animal, or the cell function may be induced, e.g., by an invasive surgical procedure such as angioplasty. Cell functions that can be suitably assayed in these methods include, e.g., vascular cell proliferation and adhesion as well as vessel remodeling.

The in vivo assays of the present invention can be modified in a number of ways as needed. For example, in certain embodiments of the present invention, the vessel subjected to analysis is assayed in vitro following removal from the animal or assayed in vivo if desired. In other embodiments, the VEGF pathway inhibitor is administered to the animal either as a sole active agent or in combination with other active compounds (e.g., probucol), including other VEGF pathway inhibitors to be tested. In most embodiments, activity of the VEGF pathway inhibitor in a given in vivo assay is compared to a suitable control (e.g., a sham-operated animal) in which the assay is conducted the same as the test assay but without administering the VEGF pathway inhibitor to the test subject. A variety of test subjects can be employed, particularly mammals such as rabbits, primates, various rodents and the like.

As noted above, the detection assays (either in vitro or in vivo) can be conducted in a wide variety of VEGF-responsive cells, tissues and organs. Further, the assays can detect useful VEGF pathway inhibitors by measuring the activity of target molecules and functions in VEGF-related pathways. Thus, the present assays can measure activity in several cell, tissue and organ settings.

Significantly, use of multiple detection assays (e.g., a combination of the in vitro and/or in vivo assays) with a single VEGF pathway inhibitor can extend the selectivity and sensitivity of detection as desired.

Such broad spectrum testing provides additional advantages. Thus, for example, in vitro assays of the invention can efficiently perform multiple analyses, thereby enhancing efficiency and probability of identifying VEGF pathway inhibitors with therapeutic capacity. This is especially useful when large numbers of compounds need to be tested. For instance, libraries of VEGF pathway inhibitors can be made by standard synthetic methods including combinatorial-type chemistry manipulations and then tested in accord with the invention.

Additionally, many of the VEGF-related steps are “downstream” of VEGF pathway, and therefore the assays include molecules and cell functions that are active downstream of VEGF pathway. Accordingly, modest but significant changes in VEGF pathway activity can be registered as readily testable signals.

According to one aspect, methods for determining the therapeutic capacity of a VEGF pathway inhibitor to reduce angiogenesis in a subject comprise determining pre-treatment levels of angiogenesis in a subject; administering a therapeutically effective amount of a VEGF pathway inhibitor to the subject; and determining a post-treatment level of angiogenesis in the subject. In one embodiment, a decrease in the angiogenesis indicated that the VEGF pathway inhibitor is efficacious. In a related embodiment, the pre-treatment and post-treatment levels of angiogenesis are determined in a diseased tissue.

Other aspects of the invention are discussed below.

### BRIEF DESCRIPTION OF THE DRAWINGS

FIG. 1 shows the effect of concentration and time dependent action of VEGF on PECAM-1 mRNA transcription and protein expression in HUVECs. (A) Cells were treated with different concentrations of VEGF (0-30 ng/ml) for 4 hrs. Total RNA was extracted from cells treated with VEGF and equal quantity of total RNA was used for real-time RT-PCR. b-actin served as internal control to check equal quantity of cDNA. (B) quantitative real-time RT-PCR analyses were performed to precisely determine the change in gene expression of PECAM-1, in HUVECs treated with VEGF (25 ng/ml) for different time points (C) Western blot analysis of PECAM-1 in HUVECs treated with various concentrations of VEGF for 4 hrs. Bottom panel shows the densitometric quantification of protein expression. (D) Western blot analysis of PECAM-1 expression to determine the time course of VEGF (25 ng/ml) on PECAM-1 expression. Bottom panel shows the densitometric quantification of protein expression. Figures shown are representative of experiments repeated in triplicate yielding similar results and the values presented in the bar graphs were mean±SD.

FIG. 2 shows that VEGF stimulates and D-PDMP inhibits LacCer/GlcCer biosynthesis and PECAM-1 expression in HUVECs. (A) Cells were metabolically labeled with [¹⁴C] palmitate (1 μCi/ml) for 24 hrs at 37° C. Next, the cells were washed and incubated for 60 min, with and without D-PDMP (20 μM). Next, VEGF (25 ng/ml) was added and incubation was continued at 37° C. At the indicated time intervals, cells were washed three times with PBS and lipids were extracted and LacCer content was determined as descried in Materials and Materials section. The control values (DMSO); vehicle treated cells) for LacCer (panel A) and GlcCer (panel B) mass were 21.76 nmol/mg protein, and 9.77 mmol/mg protein, respectively. Each point represented is a mean±S.D of three separate experiments performed in duplicate. Open spheres (□) indicate cells that were treated with VEGF (25 ng/ml) at time intervals indicated and the solid spheres (v) indicate cells that were pre-treated with D-PDMP (20 μM) followed by incubation with VEGF. (B) Western blot analysis of PECAM-1 expression in HUVECs treated with varying concentrations of D-PDMP for 90 min, followed by treatment with VEGF (25 ng/ml) for 4 hrs. n=3; * P<0.001 vs. vehicle control—PBS or DMSO; ** P<0.05 vs. VEGF (C) real-time RT-PCR analysis of PECAM-1 mRNA expression in HUVECs treated with either VEGF (25 ng/ml) for 4 hrs or LacCer (2.5 μM) and VEGF (25 ng/ml) or 4 hrs. In some experiments cells were pretreated with D-PDMP (20 μM) for 90 min, followed by VEGF/LacCer for 4 hrs. n=3; * P<0.001 vs. VEGF/VEGF+LacCer, **P<0.001 vs. vehicle control; *** P<0.05 vs. D-PDMP+VEGF.

FIG. 3 shows that LacCer specifically induces PECAM-1 expression and tube formation/angiogenesis in HUVECs. (A). PECAM-1 expression was performed to demonstrate that LacCer specifically induces PECAM-1 in HUVECs. Cells were treated with LacCer and its homologues such as DGDG, GlcCer or C₂Cer at 2.5 μM for 4 hrs. Subsequently, cell lysates were processed for Western immunoblot assay to determine PECAM-1 expression. [n=3; * P<0.001 vs. VC—vehicle control (DMSO)]. (B) Western blot analysis of PECAM-1 expression to demonstrate that LacCer specifically bypasses the inhibitory effect of D-PDMP on PECAM-1 expression (n=3, * P<0.001 vs. vehicle control; ** P<0.05 vs. D-PDMP treated cells). (C) Top panel depicts the effect of LacCer/VEGF in inducing angiogenesis in HUVECs and LacCer specifically reverses the inhibitory effect of D-PDMP on VEGF induced angiogenesis. Bottom panel shows the quantitative measurement of tube formation as described under “Experimental Procedures” section (n=3; * P<0.001 vs. vehicle control cells; ** P<0.05 vs. VEGF or LacCer treated cells and # P<0.05 vs. panel D).

FIG. 4 demonstrates the effect of PPMP on VEGF/LacCer induced PECAM-1 expression and angiogenesis in HUVECs. (A) Western immunoblot assay of PECAM-1 expression in cells with VEGF alone for 4 hrs or pre-treated with PPMP (90 min) followed by incubation with LacCer or GlcCer for 4 hrs (n=3; * P<0.001 vs. control cells; **P<0.001 vs. VEGF treated cells; *** P<0.05 vs. PPMP+GlcCer Cells). (B) Shows the effect of VEGF/LacCer in promoting angiogenesis in HUVECs and the inhibitory effect of PPMP on VEGF induced angiogenesis. HUVECs were treated with VEGF (25 ng/ml) for 4 hrs or D-PDMP (20 μM) for 90 min, followed by incubation with VEGF (25 ng/ml), GlcCer (2.5 μM) or LacCer (2.5 μM) for 4 hrs and in vitro angiogenesis assays were performed as described earlier. (n=3; * P<0.001 vs. vehicle control PBS or DMSO A; ** P<0.001 vs. VEGF or LacCer; *** P<0.05 vs. PPMP+VEGF; # P<0.001 vs. PPMP+VEGF).

FIG. 5 shows that the silencing of GalT-V expression using siRNA directed against human GalT-V. (A) Depicts immunoblot analysis performed to demonstrate the specificity of the rabbit polyclonal anti human GalT-V. Lanes 1-2, cell lysates of HUVECs and lanes 34 were loaded with CHO-K1 (MT) cells over expressing human GalT-V. (B) HUVECs were transfected with the indicated concentrations of duplex siRNA targeted against GalT-V, scrambled sequence (negative control siRNA) or OF (Oligofectamine) alone. Next, 48 hrs after transfection cells were harvested lysed, and GalT-V protein levels were analyzed by immunoblot probed with GalT-V antibody or b-actin, as shown in the figure. GalT-V expression was expressed as % control, when normalized with b-actin expression and the relative quantification values are shown below the immunoblot. (C) HUVECs were transfected with either scrambled GalT-V siRNA (100 nM) or GalT-V siRNA (100 nM). 48 hrs post transfection, cells were lysed and Cer synthase enzyme activity was performed as described in “Materials and Methods”. The data shown is representative of two identical experiments yielding similar result. (* P<0.05 vs. scrambled GalT-V siRNA or OF alone).

FIG. 6 demonstrates that silencing of GalT-V blunts VEGF induced PECAM-1 expression and angiogenesis in HUVECs. (A) Immunoblot analysis of PECAM-1 protein expression in HUVECs that were either transfected with siRNA specific for GalT-V or scrambled siRNA (negative control) and treated with or without VEGF (25 ng/ml) for 4 hrs. Values indicated in parenthesis are quantitative expression of PECAM-1 protein levels when normalized with b-actin. (B) Depicts angiogenesis assays in cells that were either transfected with GalT-V siRNA or scrambled siRNA followed by treatment with VEGF (25 ng/ml) for 4 hrs. (C) Depicts quantitative measurement of tube formation. The aforementioned experiments were repeated in duplicate and immunoblots shown are representative of them yielding reproducible results. (* P<0.05).

FIG. 7 shows that PECAM-1 is necessary for LacCer/VEGF induced angiogenesis. (A) HUVECs were pretreated with either SU 1498 for 1 hr or anti human PECAM-1 mAb for 1 hr and then exposed to VEGF (25 ng/ml)/LacCer (2.5 μM) for 4 hrs and then tube formation assays were carried out as described in the “Materials and Methods” section (B) Depicts quantitative analysis of tube formation. (n=3), * P<0.001 vs. vehicle control that received either PBS or DMSO; ** P<0.001 vs. VEGF).

FIG. 8 demonstrates that the lack of PECAM-1 fails to induce angiogenesis in vitro. REN (WT) [A] were either stimulated with VEGF (25 ng/ml) [B]; LacCer (2.5 μM) [C] or REN (rhPECAM-1) [D] with VEGF (25 ng/ml) (E) or LacCer [F] for 4 hrs and then in vitro angiogenesis assays were performed as described earlier. Results shown here are from set of experiments performed in triplicate yielding similar results.

### DETAILED DESCRIPTION OF THE INVENTION

As discussed above, the present invention features therapeutic methods for treatment and prevention of angiogenesis related conditions. The treatment methods of the invention generally include administering a therapeutically effective amount of a VEGF pathway modulator (e.g., inhibitor or activator) to a subject, preferably a patient in need of such treatment.

It also has been unexpectedly found that VEGF is a cell signaling molecule that can modulate various angiogenesis related conditions. That is, changes in cell levels of VEGF pathway members alter the development or severity of those diseases. More particularly, it has been unexpectedly found that in VEGF-responsive cells, VEGF functions as a signal molecule to effect changes in certain cell steps (sometimes referred to herein as “VEGF-related steps” or “VEGF-related pathways”). VEGF-related pathways impact a variety of functions relating to angiogenesis.

The nomenclature used herein for LacCer synthase is as follows: originally, LacCer synthase was purified and characterized from human kidney (J Biol Chem. 1982; 267: 7148-7153). Next, Nomura et al., (J Biol Chem. 1998; 273: 1357013577.) cloned rat brain LacCer synthase that was termed GalT-2/GalT-IV. Subsequently, information analyzed from the Gen Bank revealed the presence of another b 1-4 galactosyl transferase (Glycobiology. 1998; 8: 517-526) that had 68% homology to that of rat brain LacCer synthase. This LacCer synthase was termed GalT-V. Based upon biochemical and functional studies it was suggest that GalT-V is a bonafide LacCer synthase (Kolmakova A. and Chatterjee S. Glycoconjugate J. 2005 in Press). In HUVECs GalT-V is the major LacCer synthase based upon RT-PCR and Northern blot analysis (Kohmakova A. and Chatterjee S. Glycoconjugate J. 2005 in Press). Accordingly in this manuscript we have used the term GalT-V to specifically designate the HUVEC enzyme. Where we are not sure whether the enzyme is GalT-V or GalT-VI we have referred it as LacCer synthase.

“Providing a polypeptide,” refers to obtaining, by for example, buying or making the polypeptides. The polypeptides may be made by any known or later developed biochemical techniques. For example, the polypeptides may be obtained from cultured cells. The cultured cells, for example, may comprise an expression construct comprising a nucleic acid segment encoding the polypeptide.

Cells and/or subjects may be treated and/or contacted with one or more anti-angiogenic treatments including, surgery, chemotherapy, radiotherapy, gene therapy, immune therapy or hormonal therapy, or other therapy recommended or proscribed by self or by a health care provider.

As used herein, “treating, preventing or alleviating angiogenesis” refers to the prophylactic or therapeutic use of the therapeutic agents described herein, e.g., VEGF pathway inhibitors.

As used herein “angiogenesis” in some instance refers to conditions caused by or related to instances of aberrant angiogenesis. That is to aberrant increased or decreased angiogenesis. Conditions related to increased angiogenesis, include, for example, angiogenesis is related to cancer, coronary heart disease, tumor metastasis, inflammatory vascular disease, inflammation, ischemia-reperfusion injury, hypertension or diabetes. These conditions related to increased angiogenesis are refereed to herein as being treated with VEGF pathway inhibitors. Conditions related to decreased angiogenesis, include, for example, tissue degeneration is related to intrauterine growth of a fetus, systemic sclerosis, wound healing, ischemia, reperfusion injury, diabetes, coronary artery disease, tumor growth. These conditions related to decreased angiogenesis are refereed to herein as being treated with VEGF pathway activators.

“VEGF pathway” and “VEGF pathway members” as used herein, describe proteins and other signaling molecules that are responsive to VEGF stimulation of cells. For example, VEGFR, PECAM-1, LacCer synthase and PLA2.

As used herein a “reduced level” of a polypeptide, or fragments or variants thereof refers to a lower than average, expected or an actual lower value of expression for a particular cell or subject.

“Substantially purified” when used in the context of a a polypeptide, or fragment or variant thereof that are at least 60% free, preferably 75% free and more preferably 90% free from other components with which they are naturally associated. An “isolated polynucleotide” is, therefore, a substantially purified polynucleotide.

The term “subject” includes organisms which are capable of suffering from angiogenesis or who could otherwise benefit from the administration of a compound or composition of the invention, such as human and non-human animals. Preferred human animals include human patients suffering from or prone to suffering from angiogenesis or associated state, as described herein. The term “non-human animals” of the invention includes all vertebrates, e.g., mammals, e.g., rodents, e.g., mice, and non-mammals, such as non-human primates, e.g. sheep, dog, cow, chickens, amphibians, reptiles, etc.

A method for “predicting or diagnosing” as used herein refers to a clinical or other assessment of the condition of a subject based on observation, testing, or circumstances.

“Determining a level of expression” may be by any now known or hereafter developed assay or method of determining expression level, for example, immunological techniques, PCR techniques, immunoassay, quantitative immunoassay, Western blot or ELISA, quantitative RT-PCR, and/or Northern blot. The level may be of RNA or protein.

A sample or samples may be obtained from a subject, for example, by swabbing, biopsy, lavage or phlebotomy. Samples include tissue samples, blood, sputum, bronchial washings, biopsy aspirate, or ductal lavage.

“Therapeutically effective amount” as used herein refers to an amount of an agent which is effective, upon single or multiple dose administration to the cell or subject, in or in prolonging the survivability of the patient with such a disorder beyond that expected in the absence of such treatment.

Compositions described herein may be administered, for example, systemically, intratumorally, intravascularly, to a resected tumor bed, orally, or by inhalation.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. The primer must be sufficiently long to prime the synthesis of extension products in the presence of the inducing agent. The exact lengths of the primers will depend on many factors, including temperature, source of primer and the use of the method.

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Boil. Chem. 260:2605-2608 (1985); Rossolini et al., Mol. Cell. Probes, 8:91-98 (1994)). The term nucleic acid is used interchangeably with gene, cDNA, mRNA, oligonucleotide, and polynucleotide.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

As used herein, the term “polymerase chain reaction” (PCR) refers to the methods of U.S. Pat. Nos. 4,683,195, 4,683,202, and 4,965,188, all of which are hereby incorporated by reference, directed to methods for increasing the concentration of a segment of a target sequence in a mixture of genomic DNA without cloning or purification. As used herein, the terms “PCR product” and “amplification product” refer to the resultant mixture of compounds after two or mote cycles of the PCR steps of denaturation, annealing and extension are complete. These terms encompass the case where there has been amplification of one or more segments of one or more target sequences.

As used herein, the terms “restriction endonucleases” and “restriction enzymes” refer to bacterial enzymes, each of which cut double-stranded DNA at or near a specific nucleotide sequence.

As used herein, the term “recombinant DNA molecule” as used herein refers to a DNA molecule, which is comprised of segments of DNA joined together by means of molecular biological techniques.

As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, an oligonucleotide having a nucleotide sequence encoding a gene refers to a DNA sequence comprising the coding region of a gene or in other words the DNA sequence, which encodes a gene product. The coding region may be present in either a cDNA or genomic DNA form. Suitable control elements such as enhancers/promoters, splice junctions, polyadenylation signals, etc., may be placed in close proximity to the coding region of the gene if needed to permit proper initiation of transcription and/or correct processing of the primary RNA transcript. Alternatively, the coding region utilized in the vectors of the present invention may contain endogenous enhancers/promoters, splice junctions, intervening sequences, polyadenylation signals, etc., or a combination of both endogenous and exogenous control elements.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, or 95% identity over a specified region), when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Optionally, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math., 2:482 (1981), by the homology alignment algorithm of Needleman & Wunsch, J. Mol. Biol., 48:443 (1970), by the search for similarity method of Pearson & Lipman, Proc. Natl. Acad. Sci. U.S.A., 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

As used herein, the term “antibody” refers to any molecule which has specific immunoreactivity activity, whether or not it is coupled with another compound such as a targeting agent, carrier, label, toxin, or drug. Although an antibody usually comprises two light and two heavy chains aggregated in a “Y” configuration with or without covalent linkage between them, the term is also meant to include any reactive fragment or fragments of the usual composition, such as Fab molecules, Fab proteins or single chain polypeptides having binding affinity for an antigen. Fab refers to antigen binding fragments. As used herein, the term “Fab molecules” refers to regions of antibody molecules which include the variable portions of the heavy chain and/or light chain and which exhibit binding activity. “Fab protein” includes aggregates of one heavy and one light chain (commonly known as Fab), as well as tetramers which correspond to the two branch segments of the antibody Y (commonly known as F(ab)₂), whether any of the above are covalently or non-covalently aggregated so long as the aggregation is capable of selectively reacting with a particular antigen or antigen family.

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with the proteins disclosed herein. The antibodies can be tested for their desired activity using the in vitro assays described herein, or by analogous methods, after which their in vivo therapeutic and/or prophylactic activities are tested according to known clinical testing methods.

The antibodies of the instant invention are raised against VEGF pathway members, e.g., VEGF, VEGFR, VEGF pathway, PECAM-1.

The antibody can be a polyclonal, monoclonal, recombinant, e.g., a chimeric or humanized, fully human, non-human, e.g., murine, single chain antibody, or fully synthetic. Chimeric, humanized, but most preferably, completely human antibodies are desirable for applications which include repeated administration, e.g., therapeutic treatment of human patients, and some diagnostic applications. In a related embodiment, the antibody can be coupled to a toxin.

### Therapeutic Methods and Compositions

The present invention provides for both prophylactic and therapeutic methods of treating a subject having, or at risk of having, angiogenesis.

The instant invention further provides a method of treating angiogenesis in a subject, which comprises administering to the subject one or more doses of a pharmaceutical composition of the invention effective to reduce angiogenesis in a subject, thereby treating the angiogenesis.

As used herein, the term “angiogenesis-related disease” and “aberrant angiogenesis,” refer to both the asymptomatic and symptomatic phases, and to increased angiogenesis (e.g., cancer, coronary heart disease, tumor metastasis, inflammatory vascular disease, or diabetes) and decreased angiogenesis (tissue degradation).

“Aberrant angiogenesis,” as used herein refers to increased angiogenesis (e.g., cancer, coronary heart disease, tumor metastasis, inflammatory vascular disease, or diabetes) and decreased angiogenesis (tissue degradation).

As used herein, the term “treatment” is defined as the application or administration of a therapeutic agent to a patient, or application or administration of a therapeutic agent to an isolated tissue or cell line from a patient, who has, or is at risk of having, aberrant angiogenesis, with the purpose to cure, heal, alleviate, relieve, alter, remedy, ameliorate, improve or affect the infection or the symptoms of infection. A therapeutic agent includes, but is not limited to, peptides, antibodies, or fragments thereof, small molecules, lipids, and nucleic acids, as described herein.

The term “effective amount” refers to a dosage or amount that is sufficient to reduce or increase the amount of angiogenesis to result in amelioration of symptoms in a patient or to achieve a desired biological outcome, e.g., lower or higher angiogenesis.

“Pharmaceutically acceptable excipients or vehicles” include, for example, water, saline, glycerol, ethanol, etc. Additionally, auxiliary substances, such as wetting or emulsifying agents, pH buffering substances, and the like, may be present in such vehicles.

The therapeutic methods of the invention generally comprise administration of a therapeutically effective amount of a VEGF pathway inhibitor or activator to a subject in need of such treatment, such as a mammal, and particularly a primate such as a human. Treatment methods of the invention also comprise administration of an effective amount of a compound of Formula I as defined herein to a subject, particularly a mammal such as a human in need of such treatment for an indication disclosed herein.

A variety of VEGF pathway inhibitors can be employed in the present treatment methods. Simple testing, e.g., in a standard in vitro assay as defined above, can readily identify suitable VEGF pathway inhibitors. Preferred VEGF pathway inhibitors include those that contain a propanol backbone. Generally preferred for use in the treatment methods of the invention are compounds of the following Formula I:

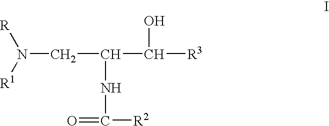

wherein R and R¹ are independently selected from the group consisting of hydrogen and straight-chained or branched C₁-C₆ alkyl with or without a substituent such as amino, hydroxy or mercapto and further wherein R and R¹ may be taken together to form a 5, 6 or 7-membered ring substituent such as pyrrolidino, morpholino, thiomorpholino, piperidino, azacycloheptyl and the like;

R² is selected from the group consisting of branched or straight-chained C₆-C₃₀ alkyl with or without one to three double bonds; and

R³ is selected from the group consisting of straight-chained or branched C₆-C₂₀ alkyl with or without one to three double bonds and aryl such as carbocyclic aryl (e.g., phenyl), or substituted aryl such as carbocyclic aryl (e.g., phenyl), where the substituent is halo, C₁-C₄ alkoxy, methylenedioxy, C₁-C₄ mercapto, amino or substituted amino in which the amino substituents may suitably be C₁-C₄ alkyl.

Suitable compounds of Formula I above and other VEGF pathway inhibitors can be readily prepared by known procedures or can be obtained from commercial sources. See, for example, Abe, A. et al., (1992) J. Biochem. 111:191-196; Inokuchi, J. et al. (1987) J. Lipid Res. 28:565-571; Shukla, A. et al. (1991) J. Lipid Res. 32:73; Vunnam, R. R. et al., (1980) Chem. and Physics of Lipids 26:265; Carson, K. et al., (1994) Tetrahedron Lets. 35:2659; and Akira, A. et al., (1995) J. Lipid Research 36:611.

VEGF pathway inhibitors also include, for example, SU-1498, Gö6976, Gö6850, bromophenacyl bromide (BMB), methyl-arachidonyl fluorophosphonate (MAFP), pyrrolidine carbodithioicacid, diphenylene iodonium chloride and N-acetyl-L-cysteine.

VEGF pathway inhibitors also include, for example, PECAM-1, LacCer, or LacCer synthase antibodies or fragments thereof. Exemplary antibodies include LacCer synthase (GalT-V/VI) antibodies specific for mitigating VEGF-induced in vitro angiogenesis/tube formation. Other exemplary antibodies for use in the methods described herein are antibodies specific for GalT-V peptide sequences including IGAQVYEQVLRSAYAKRNSSVND and IGMHMI-----RLYTNKNSTLNGT. Further antibodies useful in the methods described herein are antibodies specific for the following LacCer synthase (GalT-V/VI) sequences:

HUMAN GalT-V PEPTIDES:

| | | |
|---|---|---|
| (115) | PERLP | (119) |
| (145) | PTIKLGGHWKP | (155) |
| (160) | PRWKVAILIP | (169) |
| (169) | PFRNRHEHLP | (178) |
| (178) | PVLFRHLLP | (186) |
| (316) | PEGDTGKYKSIP | (328) |

HUMAN GalT-VI PEPTIDES

| | | |
|---|---|---|
| (97) | PENFTYSP | (104) |
| (104) | PYLP | (107) |
| (107) | PCPEKLP | (113) |
| (143) | PGGHWRP | (149) |
| (154) | PRWKVAVLIP | (163) |
| (163) | PFRNRHEHLP | (172) |
| (172 | PIFFLHLIP | (180) |
| (311) | PEGDLGKYKSIP | (322) |
| (374) | PELAP | (378). |

VEGF pathway inhibitors also include, for example, PECAM-1, LacCer, or LacCer synthase siRNA molecules. For example, 5′-CGG AGU GAG UGG CTU AAC A dTdT-3′ (sense), 5, UGU UAA GCC ACU CAC UCC G dTdT-3′ (antisense).

VEGF pathway inhibitors also include, for example, PECAM-1, LacCer, or LacCer synthase peptides or fragments thereof. Exemplary peptides include the following LacCer synthase (GalT-V/VI) peptides:

HUMAN GalT-V PEPTIDES:

| | | |
|---|---|---|
| (115) | PERLP | (119) |
| (145) | PTIKLGGHWKP | (155) |
| (160) | PRWKVAILIP | (169) |
| (169) | PFRNRHEHLP | (178) |
| (178) | PVLFRHLLP | (186) |
| (316) | PEGDTGKYKSIP | (328) |

HUMAN GalT-VI PEPTIDES

| | | |
|---|---|---|
| (97) | PENFTYSP | (104) |
| (104) | PYLP | (107) |
| (107) | PCPEKLP | (113) |
| (143) | PGGHWRP | (149) |
| (154) | PRWKVAVLIP | (163) |
| (163) | PFRNRHEHLP | (172) |
| (172 | PIFFLHLIP | (180) |
| (311) | PEGDLGKYKSIP | (322) |
| (374) | PELAP | (378) |

Other useful peptides include peptides of PLA2 because phospholipase A2 (PLA2) activation is required by LacCer to induce PECAM-1 expression (Gong, N . . . . Chatterjee, S et al Proc Natl Acad Sci USA. 101; 6490-6495 2004) and herein it is presented that PLA2 inhibitors also mitigate VEGF/LacCer induced angiogenesis in human endothelial cells. The use of a PLA2 peptide that can mitigate PLA2 activity and consequently PECAM-1 expression and angiogenesis in a subject. Exemplary sequences of a PLA2 peptide includes peptides having the sequence CC(P)-x-H-(LGY)-x-C, wherein histidine (H) is the active site of the enzyme.

Other si-RNAs and peptides are envisioned and one of skill in the art having the benefit of this disclosure (e.g., sequences and screening methods), would understand how to make and use other such si-RNA and peptide inhibitors of the VEGF pathway. For example, si-RNAs may be constructed using the following sequences: AF38663 (GalT-V), AF38664 (GalT-VI), NM_{—}008816, NM_{—}000442, (PECAM1), NM_{—}077435, NM_{—}001025370, NM_{—}001025369, NM_{—}001025368, NM_{—}001025367, NM_{—}003376, NM_{—}001025366, (VEGF), X94263, XM_{—}497921, AB065372, AJ319908, D64016, NM_{—}002019, (VEGFR) and NM_{—}000300, BC005919, (PLA2) which are hereby incorporated by reference in their entirety.

In the therapeutic methods of the invention, a treatment compound can be administered to a subject in any of several ways. For example, a VEGF pathway inhibitor or activator can be administered as a prophylactic to prevent the onset of or reduce the severity of a targeted condition. Alternatively, a VEGF pathway inhibitor can be administered during the course of a targeted condition.

A treatment compound can be administered to a subject, either alone or in combination with one or more therapeutic agents, as a pharmaceutical composition in mixture with conventional excipient, i.e. pharmaceutically acceptable organic or inorganic carrier substances suitable for parenteral, enteral or intranasal application which do not deleteriously react with the active compounds and are not deleterious to the recipient thereof. Suitable pharmaceutically acceptable carriers include but are not limited to water, salt solutions, alcohol, vegetable oils, polyethylene glycols, gelatin, lactose, amylose, magnesium stearate, talc, silicic acid, viscous paraffin, perfume oil, fatty acid monoglycerides and diglycerides, petroethral fatty acid esters, hydroxymethyl-cellulose, polyvinylpyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously react with the active compounds.

Such compositions may be prepared for use in parenteral administration, particularly in the form of liquid solutions or suspensions; for oral administration, particularly in the form of tablets or capsules; intranasally, particularly in the form of powders, nasal drops, or aerosols; vaginally; topically e.g. in the form of a cream; rectally e.g. as a suppository; etc. The VEGF pathway inhibitors or activators may also be administered via stent. Exemplary stents are described in US Patent Application Publication Nos: 20050177246; 20050171599, 20050171597, 20050171598, 20050169969, 20050165474, 20050163821, 20050165352, and 20050171593.

The pharmaceutical agents may be conveniently administered in unit dosage form and may be prepared by any of the methods well known in the pharmaceutical arts, e.g., as described in Remington's Pharmaceutical Sciences (Mack Pub. Co., Easton, Pa., 1980). Formulations for parenteral administration may contain as common excipients such as sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers may be useful excipients to control the release of certain VEGF pathway inhibitors.

Other potentially useful parenteral delivery systems include ethylene-vinyl acetate copolymer particles, osmotic pumps, implantable infusion systems, and liposomes. Formulations for inhalation administration contain as excipients, for example, lactose, or may be aqueous solutions containing, for example, polyoxyethylene-9-lauryl ether, glycocholate and deoxycholate, or oily solutions for administration in the form of nasal drops, or as a gel to be applied intranasally. Formulations for parenteral administration may also include glycocholate for buccal administration, methoxysalicylate for rectal administration, or citric acid for vaginal administration. Other delivery systems will administer the therapeutic agent(s) directly at a surgical site, e.g. after balloon angioplasty a VEGF pathway inhibitor may be administered by use of stents.

A VEGF pathway modulator (e.g., inhibitor or activator) can be employed in the present treatment methods as the sole active pharmaceutical agent or can be used in combination with other active ingredients, e.g., probucol, known antioxidants (e.g. Vitamin C or E) or other compounds. As used herein, modulator refers to an inhibitor or activator of the VEGF pathway.

The concentration of one or more treatment compounds in a therapeutic composition will vary depending upon a number of factors, including the dosage of the VEGF pathway inhibitor or activator to be administered, the chemical characteristics (e.g., hydrophobicity) of the composition employed, and the intended mode and route of administration. In general terms, one or more than one of the VEGF pathway inhibitors or activators may be provided in an aqueous physiological buffer solution containing about 0.1 to 10% w/v of a compound for parenteral administration.

It will be appreciated that the actual preferred amounts of active compounds used in a given therapy will vary according to e.g. the specific compound being utilized, the particular composition formulated, the mode of administration and characteristics of the subject, e.g. the species, sex, weight, general health and age of the subject. Optimal administration rates for a given protocol of administration can be readily ascertained by those skilled in the art using conventional dosage determination tests conducted with regard to the foregoing guidelines. Suitable dose ranges may include from about 1 μg/kg to about 100 mg/kg of body weight per day.

Therapeutic compounds of the invention are suitably administered in a protonated and water-soluble form, e.g., as a pharmaceutically acceptable salt, typically an acid addition salt such as an inorganic acid addition salt, e.g., a hydrochloride, sulfate, or phosphate salt, or as an organic acid addition salt such as an acetate, maleate, fumarate, tartrate, or citrate salt. Pharmaceutically acceptable salts of therapeutic compounds of the invention also can include metal salts, particularly alkali metal salts such as a sodium salt or potassium salt; alkaline earth metal salts such as a magnesium or calcium salt; ammonium salts such an ammonium or tetramethyl ammonium salt; or an amino acid addition salts such as a lysine, glycine, or phenylalanine salt.

Preferred VEGF pathway modulators (e.g., inhibitors and activators) exhibit significant activity in a standard cell proliferation assays. Preferably, the VEGF pathway inhibitor inhibits cell proliferation by at least 15 or 25%, preferably at least 50%, relative to a suitable control assay. Preferably, the VEGF pathway activator activates cell proliferation by at least 15 or 25%, preferably at least 50%, relative to a suitable control assay. In such an assay, between about 0.1 to 100 μM, preferably between about 1 to 50 μM of a desired VEGF pathway inhibitor or activator is used. Exemplary cell proliferation assays include counting viable cells and monitoring activity of specified citric acid cycle enzymes such as lactate dehydrogenase. A preferred assay measures incorporation of one or more detectably-labeled nucleosides into DNA, e.g., by:

a) culturing suitable cells in medium and adding
1) a candidate VEGF pathway inhibitor or activator and
2) a radiolabeled nucleoside such as ³H-thymidine typically in an amount between about 0.1 to 100 μCi;

b) incubating the cells, e.g., for about 6-24 hours, and typically followed by washing; and

c) measuring incorporation of the radiolabeled nucleoside into DNA over that time relative to a control culture that is prepared and incubated under the same conditions as the assay culture but does not include the potential VEGF pathway inhibitor or activator. The measurement can be achieved by several methods including trichloroacetic acid (TCA) precipitation of labeled DNA on filters followed by scintillation counting. See e.g., Chatterjee, S., Biochem. Biophys. Res Comm. (1991) 181:554; Chatterjee, S. et al. (1982) Eur. J. Biochzem. 120:435 for disclosure relating to this assay.

References herein to a “standard in vitro cell proliferation assay” or other similar phrase refer to an assay that includes the above steps a) through c). One preferred example of a cell proliferation assay uses aortic smooth muscle cells (ASMCs), particularly those obtained from a human, cow or a rabbit. A suitable protocol involves preparing ASMCs according to standard methods and culturing same in microtitre plates in a suitable medium such as Ham's F-10. A desired VEGF pathway inhibitor or activator is then diluted in the medium, preferably to a final concentration of between about 1 to 100 μg, more preferably between about 1 to 50 μg per ml of medium or less followed by an incubation period of between about 1-5 days, preferably about 1 day or less. Following the incubation, a standard cell proliferation can be conducted, e.g., incorporation of tritiated thymidine or lactate dehydrogenase assay as mentioned above. The assays are preferably conducted in triplicate with a variation of between 5% to 10%. See e.g., Ross, R. J. Cell. Biol. (1971) 50:172; Chatterjee, S. et al. (1982) Eur. J. Biochem. 120:435; Bergmeyer, H. V. In Principles of Enzymatic Analysis. (1978) Verlag Chemie, NY.

Additionally, preferred VEGF pathway inhibitors or activators exhibit significant activity in a conventional cell adhesion assay. Preferably, the VEGF pathway inhibitor inhibits cell adhesion by at least 25%, preferably at least 50% or more relative to a suitable control assay. Preferably, the VEGF pathway activator activates cell adhesion by at least 25%, preferably at least 50% or more relative to a suitable control assay. In such an assay, between about 0.1 to 100M, preferably between about 1 to 50 μM of a desired VEGF pathway inhibitor or activator is used. For example, a preferred cell adhesion assay includes the following steps:

a) labeling a first population of immune cells, preferably certain leukocytes, with a detectable label which can be a chromatic, radioactive, luminescent (e.g., fluorescent, or phosphorescent), or enzymatic label capable of producing a detectable label,

b) contacting the first population of cells with a second population of endothelial cells detectably-labeled, e.g., with a chromatic, radioactive, luminescent (e.g., fluorescent or phosphorescent), or enzymatic label preferably different from the label employed in step a); and

c) detecting any adhesion between the first and second population of cells.

References herein to a “standard in vitro cell adhesion assay” or other similar phrase refer to an assay that includes the above steps a) through c). The detection in step c) can be achieved by a variety of methods such as microscopy, particularly confocal microscopy and fluorescence-based photomicroscopy involving FACS; automated cell sorting techniques, immunological methods such as ELISA and RIA; and scintillation counting. See examples below for disclosure relating to preferred cell adhesion assays.

A preferred in vitro cell adhesion assay measures polymorphonuclear leukocytes (PMNs and/or myocytes) or platelets and increased endothelial cell adhesion before, during or after contact with a desired VEGF pathway inhibitor or activator. The PMNs or myocytes can be collected and purified according to standard methods detailed below. The PMNs or myocytes are then labeled by incubation with a suitable fluorescent dye such as fluorescent Cell Tracker dye (e.g., green) or Calcein-AM. At about the same time, an endothelial cell monolayer prepared in accordance with standard cell culture methods on a suitable substrate such as a slide or a sterilized plastic petri dish is contacted by the VEGF pathway inhibitor or activator and labeled with another fluorescent dye such as fluorescent Cell Tracker dye (e.g., orange). The PMNs or myocytes and endothelial cells are then incubated for between about 10 minutes to a few hours, preferably about 30 minutes at 37° C. Non-adherent cells are then washed away from the slide with a physiologically acceptable buffer such as phosphate-buffered saline (PBS). Adhering cells are then quantitated by standard methods such as by use of a fluorescence plate reader. The number of adherent cells on the slide can be quantitated in several ways including expressing the number of PMN/mm² on the endothelial cell monolayer. Alternatively, the adhering cells can be quantitated by inspection following photomicroscopy visualized and photographed by microscopy. Cell adherence is then evaluated by inspection of the photomicrograph. See the examples which follow.

Particularly preferred are GalT-V assays conducted with the ASMCs and performed in accordance with previously described methods. See e.g., Chatterjee, S., and Castiglione, E. (1987) Biochem. Biophys. Acta, 923:136; and Chatterjee, (1991) S. Biochem. Biophys. Res Comm., 181:554.

Additionally preferred in vitro cell adhesion assays include immunological detection of adhesion molecules on PMNs using specified antibodies, particularly monoclonals, capable of specifically binding the adhesion molecules. A particularly preferred assay involves flow cytometry.

The in vitro adhesion assays described above are compatible with analysis of a variety of specified adhesion molecules such as ICAM-1 (intracellular adhesion molecule 1), Mac-1 (CD11b/CD18), LFA-1 and selectin.

Another preferred assay of the invention includes the following steps a) through d):
a) culturing a population of VEGF-responsive cells preferably to confluency in lipoprotein-deficient serum medium, e.g., about 1 mg lipoprotein-deficient serum/protein/ml of medium or less;
b) harvesting the cells preferably in a suitable dispersive buffer, e.g., cacodylate buffer;
c) incubating the harvested cells preferably with a detectably labeled molecule such as a detectably-labeled nucleoside diphosphate sugar donor such as [¹⁴C]-UDP-galactose typically in an amount between about 0.1 to 100 μCi; and
d) measuring LacCer formation as indicative of the activity of the VEGF pathway enzyme.

In most instances, the assays generally described above will use known VEGF-responsive cells and will be cultured in a medium suitable for maintaining those cells in the assay, e.g., Eagles' minimum essential medium REM) or Ham's F-10 medium.

Further preferred VEGF pathway inhibitors and activators include those that exhibit at least a 2- to 5-fold greater inhibition or activation of VEGF pathway members as measured by VEGF pathway enzyme assays or expression of the gene or protein of the pathway members. More preferred are those VEGF pathway inhibitors and activators that exhibit at least about 5- to 10-fold greater inhibition or activation, and even more preferably at least about 10- to 50-fold inhibition or activation. Methods for measuring expression are described herein in the Examples.

Particularly preferred VEGF pathway inhibitors include those that are capable of specifically inhibiting one or more VEGF pathway enzymes. That is, the identified VEGF pathway inhibitor is a relatively poor inhibitor of other enzymes. Significantly, the VEGF pathway inhibitor should avoid undesired pharmacological effects that could arise from non-selective inhibition of other VEGF-related enzymes.

The in vivo assays of the invention are particularly useful for subsequent evaluation of VEGF pathway inhibitors and activators exhibiting suitable activity in an in vitro assay. A rabbit model of restenosis accompanying an invasive surgical procedure such as balloon angioplasty is preferred. One suitable protocol involves administering to the rabbit a suitable vehicle or vehicle combined with one or more VEGF pathway inhibitors of interest. The amount of the VEGF pathway inhibitor administered will vary depending on several parameters including the extent of damage associated with the surgical procedure of interest. In instances where balloon angioplasty is employed, the rabbit will typically receive a candidate VEGF pathway inhibitor in a dose (e.g., i.m. or i.p.) of between about 0.5 to 100, preferably 1 to 20 and more preferably about 10 mg/kg body weight of the rabbit. A preferred dosage schedule provides for administration of a VEGF pathway inhibitor starting 24 hours prior to conducting an invasive surgical procedure, and then continuing administration of the VEGF pathway inhibitor for 15 days following the surgical procedure. In other protocols, daily injections of the VEGF pathway inhibitor may be made for about 2 to 12 weeks following the invasive surgical procedure. Daily injections, e.g., i.m. or i.p., of the VEGF pathway inhibitor are generally preferred. Subsequently, the rabbits are euthanized and a vessel removed for examination, preferably the aorta. The vessel is then fixed with formalin and analyzed for proliferation of vascular endothelia, media and advantitia using standard histological procedures.

The term “invasive surgical procedure” means a medical or veterinary technique associated with significant damage to the endothelium of a vessel impacting, e.g., an organ such as the heart, liver or the kidney, or a limb. Such a vessel comprises the aorta, coronary vessel, femoral and iliac arteries and veins. The invasive surgical procedure can be associated with techniques involving, e.g., cardiac surgery, abdominothoracic surgery, arterial surgery, deployment of an implementation (e.g., a vascular stent or catheter), or endarterectromy (Exemplary stents and catheters, as well as method of use thereof are described in US Patent Application Publication Nos: 20050177246; 20050171599, 20050171597, 20050171598, 20050169969, 20050165474, 20050163821, 20050165352, and 20050171593). A preferred invasive surgical procedure is angioplasty, particularly balloon angioplasty. Preferably, the invasive surgical procedure is performed on a mammal such as a primate, particularly a human, rodent or a rabbit, or a domesticated animal such as a pig, dog or a cat.

Other screening methods for VEGF pathway inhibitors and activators includes:

1) culturing human umbilical vein endothelial cells (HUVEC) and/or human mesoendothelioma cell line [(REN-wild type (WT)] which lacks endogenous PECAM-1 expression and/or REN (mt-rhPECAM-1) expressing human PECAM-1;

2) contacting the cells with a candidate VEGF pathway inhibitor or activator;

3) analyzing the cells for LacCer level and/or for the expression of one or more of LacCer synthase, PECAM-1, PLA2, VEGF or VEGFR.

The expression of the genes and proteins of LacCer, LacCer synthase, PECAM-1, PLA2, VEGF or VEGFR may be by real-time PCR, PCR, reverse transcriptase PCR, Western blot, and other methods known to those of skill in the art.

Exemplary primer sequences for PECAM-1 are as follows: (forward) 5′ TGACCCTTCTGCTCTGTT 3′ and (reverse) 5′ TGAGAGGTGGTGCTGACATC 3′ respectively. β-actin primers may include, for example, (forward) 5′ AGGTCATCACTATTGGCAACGA 3′ and (reverse) 5′ CACTTCATGATGGAATTGAATGTAGTT 3′ respectively.

Methods for determining the therapeutic capacity of a VEGF pathway inhibitor to reduce angiogenesis in a subject comprise determining pre-treatment levels of angiogenesis in a subject; administering a therapeutically effective amount of a VEGF pathway inhibitor to the subject; and determining a post-treatment level of angiogenesis in the subject. In one embodiment, a decrease in the angiogenesis indicated that the VEGF pathway inhibitor is efficacious. In a related embodiment, the pre-treatment and post-treatment levels of angiogenesis are determined in a diseased tissue.

A method of assessing the therapeutic capacity or efficacy of the treatment in a subject includes determining the pre-treatment level of levels of angiogenesis by methods well known in the art (e.g., expression level of VEGF pathway members, physical diagnosis, visual inspection of tissue, measurement of tumor regression or growth at various times before, during and after treatment, wherein the measurement is with, for example, a caliper) and then administering a therapeutically effective amount of a VEGF pathway inhibitor or activator to the subject. After an appropriate period of time (e.g., after an initial period of treatment) after the administration of the compound, e.g., 2 hours, 4 hours, 8 hours, 12 hours, or 72 hours, the level of angiogenesis is determined again. The modulation of the angiogenesis indicates efficacy of the treatment. The level of angiogenesis may be determined periodically throughout treatment. For example, the angiogenesis may be checked every few hours, days or weeks to assess the further efficacy of the treatment. A decrease in angiogenesis indicates that the treatment with an inhibitor is efficacious. The method described may be used to screen or select subject that may benefit from treatment with a VEGF pathway inhibitor.

According to the methods described herein, the diseased tissue is one or more of lung, heart, liver, tumor, or vasculature. The level of angiogenesis may be determined by PECAM-1 expression, GatT-V expression, tube formation, or LacCer level.

As noted above, the present invention includes methods of detecting and analyzing VEGF pathway inhibitors and activators with therapeutic capacity to treat or prevent angiogenesis related conditions. The VEGF pathway activity can be measured by methods referenced herein.

Generally stated, the novel VEGF-related steps disclosed herein have been found to relate changes in VEGF pathway activity to angiogenesis related conditions. Detection methods of the invention are formatted to include one or more VEGF pathway members. More particularly, the detection methods include specific steps that measure the activity of molecules which act to modulate cell angiogenesis.

The VEGF-related steps are found in cells responsive to VEGF. A VEGF-responsive cell can be an immortalized cell line or primary culture of cells (e.g., obtained form a tissue or organ) that manifests a change in one or more specific cell molecules or functions such as angiogenesis following contact with a suitable amount of VEGF.

More specifically, one or a combination of strategies can identify a VEGF-responsive mammalian cell. For example, in one approach, about 1×10⁵ cells are seeded in petri dishes in suitable growth medium. For primary cultures of cells, a desired tissue or organ is obtained from an animal and dispersed according to standard methods (e.g., by sonication, mechanical agitation, and/or exposure to dispersing agents known in the field, e.g., detergents and proteases). After one or a few days, the growth medium is removed from the petri dish and the cells washed with phosphate-buffered saline. The cells are then primed in a suitable medium for about 1 to 5 hours at which pointVEGF is added to culture. The amount of VEGF added will depend on several parameters such as the particular cell or tissue type being tested. In most cases however, the VEGF will be added to the culture at a concentration of between about 1 μg to 1 mg, preferably between about 1 μg to 500 μg, and more preferably between about 1 μg to 50 μg per ml of culture medium. After exposing the cells to the VEGF for between about 1 to 60 minutes, preferably between about 1 to 10 minutes or less, the medium is removed and the cells lysed in an appropriate lysis buffer such as those described in detail below. The cells are then assayed according to any of the methods described herein for response to the added VEGF.

Particularly preferred VEGF-responsive mammalian cells include those cells associated with vascular endothelium, e.g., cells associated with the vasculature of an organ or limb, particularly heart or kidney cells. More particularly, human umbilical vein endothelial cells (HUVEC) and endothelial cells.

Preferred VEGF pathway inhibitors also include those that exhibit good capacity to modulate one or more specified molecules in a VEGF-related pathway following exposure to VEGF. Particularly preferred compounds exhibit at least 20%, preferably at least 50% and more preferably at least 90% or more of a decrease or increase in the activity of the molecule (relative to a suitable control assay) at a concentration of between about 0.1 to 100 μg/ml, preferably between about 1 to 10 μg/ml in an in vitro detection assay. The activity of the molecules can decrease or increase in any of several readily detectable ways including altered synthesis, degradation or storage; protein modification, e.g., phosphorylation, or through an allosteric effect as with certain enzymes.

In particular, if the molecule of interest is an enzyme, preferred VEGF pathway inhibitors include those that exhibit good activity in an enzyme assay as described below. Preferably, an IC₅₀ in such an assay is about 20 μM or less, more preferably an IC₅₀ about 1 μM or less.

A control experiment is generally tailored for use in a particular assay. For example, most control experiments involve subjecting a test sample (e.g., a population of VEGF-responsive cells or lysate thereof) to medium, saline, buffer or water instead of a potential VEGF pathway inhibitor in parallel to the cells receiving an amount of test compound. A desired assay is then conducted in accordance with the present methods. Specific examples of suitable control experiments are described below.

The present detection methods also can be used to identify VEGF pathway inhibitors or activators obtained from biological sources, including specified growth factors, cytokines, and lipoproteins that modulate VEGF pathway activity.

The present detection methods further include assays which measure the activity of specified molecules in VEGF-related biochemical steps. The measurements can be conducted by standard laboratory manipulations such as chemiluminescence tests, thin layer chromatography (TLC) separations, nucleic acid isolation and purification, SDS-PAGE gel electrophoresis, autoradiography, scintillation counting, densitometery, Northern and Western Blot hybridization, and immunoassays (e.g., RIA and ELISA tests). See generally Sambrook et al. in Molecular Cloning: A Laboratory Manual (2d ed. 1989); and Ausubel et al. (1989), Current Protocols in Molecular Biology, John Wiley & Sons, New York for discussion relating to many of the standard methods, the disclosures of which are incorporated herein by reference.

In one aspect, the present in vitro assays measure the activity of certain enzymes in VEGF-responsive cells. The activity of the enzymes has been found to be modulated following exposure of the cells to VEGF and/or a specified VEGF pathway inhibitor such as PDMP or others described infra.

Additional in vitro assays are provided which measure one or more enzymes that have been found to be modulated by VEGF pathway inhibitors disclosed herein.

For example, incorporation of a nucleoside triphosphate, particularly a cyclic nucleoside triphosphate such as guanidine nucleoside triphosphate (GTP) into an oncogene protein such as the ras protein (e.g., ras-GTP loading) by the ras-GTP-binding protein can be measured by a number of distinct approaches including direct detection of nucleoside triphosphate (e.g., GTP) incorporation into Ras. For example, in one approach, VEGF-responsive cells are metabolically labeled with radioactive orthophosphate (e.g., ³²P-labeled) to detectably-label the GTP inside the cells. The labeled cells are incubated with LacCer followed by a VEGF pathway inhibitor and then washed and lysed in a suitable lysis buffer such as RIPA (see below). Subsequently, the cell lysate is separated on suitable TLC plates. The TLC plates are exposed to X-ray film and then subjected to densitometry, if desired, to quantitate incorporation of the GTP into the Ras protein. A preferred method for detecting ras-GTP loading has been disclosed in Chatterjee, S. et al., (1997) Glycobiology, 7:703.

Methods are also provided for measuring the activity of the VEGF pathway enzymes. For example, in one approach, the VEGF-responsive cells are incubated with VEGF and a potential VEGF pathway inhibitor, washed, and then harvested after about 1 to 60 minutes, preferably 1 to 10 minutes or less, after exposure to the VEGF. Whole cell lysates are prepared and then subjected to standard SDS-PAGE gel electrophoresis. The gels are transferred to a suitable membrane support and then probed with antibodies directed to the VEGF pathway members in accordance with Western blot hybridization procedures.

Additional in vitro suitable for measuring modulation by VEGF and VEGF pathway inhibitors include monitoring expression of cell proliferation factors. A preferred proliferating cell factor for such analysis is proliferating cell nuclear antigen (PCNA). In one suitable approach, the cultured cells are incubated with VEGF followed by a VEGF pathway inhibitor and then washed with a suitable buffer. PCNA in the cultured cells can be detected (and quantified if desired) by using a monoclonal antibody that is capable of specifically binding the PCNA (e.g., PC10 antibody). See Sasaki, K., et al. (1993) Cytometry 14:876-882. The PCNA then can be detected in the cells by a variety of immunological methods including flow cytometry or immunohistochemical visualization of fixed cell sections.

Vascular endothelial growth factor (VEGF) has been implicated in angiogenesis associated with coronary heart disease, vascular complications in diabetes, inflammatory vascular diseases and tumor metastasis. However, the mechanism of VEGF driven angiogenesis involving glycosphingolipids such as lactosylceramide (LacCer) is not known. To demonstrate the involvement of LacCer in VEGF induced angiogenesis, we used siRNA mediated silencing of LacCer synthase expression (GalT-V/VI) in HUVECs. This gene silencing markedly inhibited VEGF induced PECAM-1 expression and angiogenesis. Second, we used D-threo-1-phenyl-2-decanoylamino-3-morpholino-1-propanol (D-PDMP), an inhibitor of LacCer synthase and glucosylceramide synthase that significantly mitigated VEGF induced PECAM-1 expression and angiogenesis. Interestingly, these phenotypic changes were reversed by LacCer but not by structurally related compounds such as glucosylceramide, digalactosylceramide and ceramide. In a human endothelial cell line (REN) which lacks the endogenous expression of PECAM-1, VEGF/LacCer failed to stimulate PECAM-1 expression and tube formation/angiogenesis. However, in REN cells expressing human PECAM-1 gene/protein, both VEGF and LacCer induced PECAM-1 protein expression and tube formation/angiogenesis. In fact, VEGF but not LacCer induced angiogenesis were mitigated by SU-1498, a VEGF receptor tyrosine kinase inhibitor. Also, VEGF/LacCer induced PECAM-1 expression and angiogenesis was mitigated by protein kinase C (PKC) and phospholipase A2 (PLA2) inhibitors. Further, VEGF/LacCer induced PECAM-1 expression was inhibited by 1-pyrrolidinecarbodithioicacid (PDTC) an NF-kB inhibitor, diphenylene iodonium (DPI) NADPH oxidase inhibitor and N-Acetyl-L-cysteine (NAC) an antioxidant. These results indicate that LacCer generated in VEGF-treated endothelial cells may serve as an important signaling molecule for PECAM-1 expression and in angiogenesis. This finding and the reagents developed in our report may be useful as anti-angiogenic drugs for further studies in vitro and in vivo.

### Antibodies

Antibodies useful in the methods described herein are antibodies specific for VEGF pathway members, including, VEGF, VEGFR, LacCer synthase, LacCer, PECAM-1, and PLA2. Especially preferred antibodies are those which inhibit the activity of a VEGF pathway member. Methods of generating antibodies useful in the methods described herein are described more fully below.

Exemplary antibodies include LacCer synthase (GalT-V/VI) antibodies specific for mitigating VEGF-induced in vitro angiogenesis/tube formation. Other exemplary antibodies for use in the methods described herein are antibodies specific for GalT-V peptide sequences including IGAQVYEQVLRSAYAKRNSSVND and IGMHMI-----RLYTNKNSTLNGT. Further antibodies useful in the methods described herein are antibodies specific for the following LacCer synthase (GalT-V/VI) sequences:

HUMAN GalT-V PEPTIDES:

| | | |
|---|---|---|
| (115) | PERLP | (119) |
| (145) | PTIKLGGHWKP | (155) |
| (160) | PRWKVAILIP | (169) |
| (169) | PFRNRHEHLP | (178) |
| (178) | PVLFRHLLP | (186) |
| (316) | PEGDTGKYKSIP | (328) |

HUMAN GalT-VI PEPTIDES

| | | |
|---|---|---|
| (97) | PENFTYSP | (104) |
| (104) | PYLP | (107) |
| (107) | PCPEKLP | (113) |
| (143) | PGGHWRP | (149) |
| (154) | PRWKVAVLIP | (163) |
| (163) | PFRNRHEHLP | (172) |
| (172 | PIFFLHLIP | (180) |
| (311) | PEGDLGKYKSIP | (322) |
| (374) | PELAP | (378). |

Chimeric and humanized monoclonal antibodies, comprising both human and non-human portions, can be made using standard recombinant DNA techniques. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240: 1041-1043; Liu et al. (1987) Proc. Natl. Acad. Sci. USA 84: 3439-3443; Liu et al. (1987) J. Immunol. 139: 3521-3526; Sun et al. (1987) Proc. Natl. Acad. Sci. USA 84: 214-218; Nishimura et al. (1987) Canc. Res. 47: 999-1005; Wood et al. (1985) Nature 314: 446-449; and Shaw et al. (1988) J. Natl Cancer Inst. 80: 1553-1559); Morrison, S. L. (1985) Science 229: 1202-1207; Oi et al. (1986) BioTechniques 4: 214; Winter U.S. Pat. No. 5,225,539; Jones et al. (1986) Nature 321: 552-525; Verhoeyan et al. (1988) Science 239: 1534; and Beidler et al. (1988) J. Immunol. 141: 4053-4060.

Completely human antibodies are particularly desirable for therapeutic treatment of human patients. Such antibodies can be produced using transgenic mice that are incapable of expressing endogenous immunoglobulin heavy and light chains genes, but which can express human heavy and light chain genes. See, for example, Lonberg and Huszar (1995) Int. Rev. Immunol. 13: 65-93); and U.S. Pat. Nos. 5,625,126; 5,633,425; 5,569,825; 5,661,016; and 5,545,806. In addition, companies such as Abgenix, Inc. (Fremont, Calif.) and Medarex, Inc. (Princeton, N.J.), can be engaged to provide human antibodies directed against a selected antigen using technology similar to that described above.

Completely human antibodies that recognize a selected epitope can be generated using a technique referred to as “guided selection.” In this approach a selected non-human monoclonal antibody, e.g., a murine antibody, is used to guide the selection of a completely human antibody recognizing the same epitope. This technology is described by Jespers et al. (1994) Bio/Technology 12: 899-903).

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567; and Morrison et al., Proc. Natl. Acad. Sci. USA 81: 6851-6855 (1984)).

The present monoclonal antibodies can be made using any procedure which produces monoclonal antibodies. For example, monoclonal antibodies of the invention can be prepared using hybridoma methods, such as those described by Kohler and Milstein, Nature, 256: 495 (1975). In a hybridoma method, a mouse or other appropriate host animal is typically immunized with an immunizing agent to elicit lymphocytes that produce antibodies that will specifically bind to the immunizing agent.

The monoclonal antibodies also can be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of antibodies). Libraries of antibodies or active antibody fragments also can be generated and screened using phage display techniques, e.g., as described in U.S. Pat. No. 5,804,440 to Burton et al. and U.S. Pat. No. 6,096,551 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in International Patent Application Publication No. WO 94/29348, published Dec. 22, 1994, and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-lining antigen.

As used herein, the term “antibody or fragments thereof” encompasses chimeric antibodies and hybrid antibodies, with dual or multiple antigen or epitope specificities, single chain antibodies and fragments, such as F(ab′)2, Fab′, Fab, scFv and the like, including hybrid fragments. Thus, fragments of the antibodies that retain the ability to bind their specific antigens are provided. For example, fragments of antibodies which maintain HIV gp120 binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York (1988)). Also included within the meaning of “antibody or fragments thereof” are conjugates of antibody fragments and antigen binding proteins (single chain antibodies) as described, for example, in U.S. Pat. No. 4,704,692, the contents of which are hereby incorporated by reference.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase bio-longevity, to alter secretory characteristics; etc. In any case, the antibody or antibody fragment must possess a bioactive property, such as specific binding to its cognate antigen. Functional or active regions of the antibody or antibody fragment can be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment (Zoller, M. J. Curr. Opin. Biotechnol. 3: 348-354 (1992)).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods of the invention serves to lessen the chance that an antibody administered to a human will evoke an undesirable immune response.

Human antibodies also can be prepared using any other technique. Examples of techniques for human monoclonal antibody production include those described by Cole et al. (Monoclonal Antibodies and Cancer Therapy, Alan R. Liss, p. 77 (1985)) and by Boerner et al. (J. Immunol. 147(1): 86-95 (1991)). Human antibodies (and fragments thereof) also can be produced using phage display libraries (Hoogenboom et al., J. Mol. Biol. 227: 381 (1991); Marks et al., J. Mol. Biol. 222: 581 (1991)).

Human antibodies also can be obtained from transgenic animals. For example, transgenic, mutant mice that can produce a full repertoire of human antibodies in response to immunization have been described (see, e.g., Jakobovits et al., Proc. Natl. Acad. Sci. USA 90: 2551-255 (1993); Jakobovits et al., Nature 362: 255-258 (1993); and Bruggermann et al., Year in Immunol. 7: 33 (1993)). Specifically, the homozygous deletion of the antibody heavy chain joining region (J(H) gene in these chimeric and germ-line mutant mice results in complete inhibition of endogenous antibody production, and the successful transfer of the human germ-line antibody gene array into such germ-line mutant mice results in the production of human antibodies upon antigen challenge.

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fv, Fab, Fab′, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature 321: 522-525 (1986); Reichmann et al., Nature 332: 323-327 (1988); and Presta, Curr. Opin. Struct. Biol. 2: 593-596 (1992)).

Methods for humanizing non-human antibodies are well-known in the art. For example, humanized antibodies can be generated according to the methods of Winter and co-workers (Jones et al., Nature 321: 522-525 (1986); Riechmann et al., Nature 332: 323-327 (1988); and Verhoeyen et al., Science 239: 1534536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

### Pharmaceutical Compositions and Kits

The small molecule, peptide, nucleic acid, and antibody therapeutics described herein may be formulated into pharmaceutical compositions and be provided in kits. The pharmaceutical formulations may also be coated on medical devices or onto nano-particles for delivery.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, .alpha.-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical, transdermal, buccal, sublingual, intramuscular, intraperitoneal, rectal, vaginal and/or parenteral administration. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an antibody or complex of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluent commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert dilutents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

The preparations of the present invention may be given orally, parenterally, topically, or rectally. They are of course given by forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

The compounds may be administered to humans and other animals for therapy by any suitable route of administration, including orally, nasally, as by, for example, a spray, rectally, intravaginally, parenterally, intracistemally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound that is the lowest dose effective to produce a therapeutic effect. Such an effective dose will generally depend upon the factors described above. Generally, intravenous and subcutaneous doses of the compounds of this invention for a patient, when used for the indicated analgesic effects, will range from about 0.0001 to about 100 mg per kilogram of body weight per day, more preferably from about 0.01 to about 50 mg per kg per day, and still more preferably from about 1.0 to about 100 mg per kg per day. An effective amount is that amount treats angiogenesis or associated disease.

If desired, the effective daily dose of the active compound may be administered as one dose or as, two, three, four, five, six or more sub-doses administered separately at appropriate intervals throughout the day, optionally, in unit dosage forms.

While it is possible for a compound of the present invention to be administered alone, it is preferable to administer the compound as a pharmaceutical composition. Moreover, the pharmaceutical compositions described herein may be administered with one or more other active ingredients that would aid in treating a subject having a HIV infection. In a related embodiment, the pharmaceutical compositions of the invention may be formulated to contain one or more additional active ingredients that would aid in treating a subject having a HIV infection or associated disease or disorder.

The antibodies and complexes, produced as described above, can be provided in kits, with suitable instructions and other necessary reagents, in order to conduct immunoassays as described above. The kit can also contain, depending on the particular immunoassay used, suitable labels and other packaged reagents and materials (i.e. wash buffers and the like). Standard immunoassays, such as those described above, can be conducted using these kits. The pharmaceutical compositions can be included in a container, pack, kit or dispenser together with instructions, e.g., written instructions, for administration, particularly such instructions for use of the antibody or complex to treat or prevent angiogenesis or associated disease. The container, pack, kit or dispenser may also contain, for example, one or more additional active ingredients that would aid in treating a subject having aberrant angiogenesis.

RNAi Compositions for Targeting LacCer Synthase mRNA

VEGF pathway inhibitors also include, for example, PECAM-1, LacCer, LacCer synthase, or PLA2 siRNA molecules. For example, 5′-CGG AGU GAG UGG CUU AAC A dTdT-3′ (sense), 5, UGU UAA GCC ACU CAC UCC G dTdT-3′ (antisense). RNAi molecules may interfere with any portion of the mRNA of any one of a VEGF pathway member.

As used herein, the term “RNA interference” (“RNAi”) refers to a selective intracellular degradation of RNA. RNAi occurs in cells naturally to remove foreign RNAs (e.g., viral RNAs). Natural RNAi proceeds via fragments cleaved from free dsRNA, which directs the degrading mechanism to other similar RNA sequences. Alternatively, RNAi can be initiated by the hand of man, for example, to silence the expression of target genes. RNAi molecules useful for RNAi are sometime referred to herein as small interfering RNAs (siRNA).

By “reduce or inhibit” is meant the ability to cause an overall decrease preferably of 20% or greater, more preferably of 50% or greater, and most preferably of 75% or greater, in the level of protein or nucleic acid, detected by the aforementioned assays (see “expression”), as compared to samples not treated with antisense nucleotide oligomers or dsRNA used for RNA interference.

An siRNA having a “sequence sufficiently complementary to a target mRNA sequence to direct target-specific RNA interference (RNAi)” means that the ss-siRNA has a sequence sufficient to trigger the destruction of the target mRNA by the RNAi machinery or process.

Various methodologies of the instant invention include step that involves comparing a value, level, feature, characteristic, property, etc. to a “suitable control”, referred to interchangeably herein as an “appropriate control”. A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to performing an RNAi methodology, as described herein. For example, a transcription rate, mRNA level, translation rate, protein level, biological activity, cellular characteristic or property, genotype, phenotype, etc. can be determined prior to introducing a siRNA of the invention into a cell or organism. In another embodiment, a “suitable control” or “appropriate control” is a value, level, feature, characteristic, property, etc. determined in a cell or organism, e.g., a control or normal cell or organism, exhibiting, for example, normal traits. In yet another embodiment, a “suitable control” or “appropriate control” is a predefined value, level, feature, characteristic, property, etc.

An RNAi agent having a strand which is “sequence sufficiently complementary to a target mRNA sequence to direct target-specific RNA interference (RNAi)” means that the strand has a sequence sufficient to trigger the destruction of the target mRNA by the RNAi machinery or process.

By “small interfering RNAs (siRNAs)” (also referred to in the art as “short interfering RNAs”) is meant an isolated RNA molecule comprising between about 10-50 nucleotides (or nucleotide analogs), which is capable of directing or mediating RNA interference. The siRNA is preferably greater than 10 nucleotides in length, more preferably greater than 15 nucleotides in length, and most preferably greater than 19 nucleotides in length that is used to identify the target gene or mRNA to be degraded. A range of 19-25 nucleotides is the most preferred size for siRNAs. siRNAs can also include short hairpin RNAs in which both strands of an siRNA duplex are included within a single RNA molecule. siRNA includes any form of dsRNA (specifically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides. Such alterations can include the addition of non-nucleotide material, such as to the end(s) of the 21 to 23 nt RNA or internally (at one or more nucleotides of the RNA). In a preferred embodiment, the RNA molecules contain a 3′ hydroxyl group. Nucleotides in the RNA molecules of the present invention can also comprise non-standard nucleotides, including non-naturally occurring nucleotides or deoxyribonucleotides. Collectively, all such altered RNAs are referred to as analogs of RNA. siRNAs of the present invention need only be sufficiently similar to natural RNA that it has the ability to mediate RNA interference (RNAi). RNAi agents of the present invention can also include small hairpin RNAs (shRNAs), and expression constructs engineered to express shRNAs. Transcription of shRNAs is initiated at a polymerase III (pol III) promoter, and is thought to be terminated at position 2 of a 4-5-thymidine transcription termination site. Upon expression, shRNAs are thought to fold into a stem-loop structure with 3′ UU-overhangs; subsequently, the ends of these shRNAs are processed, converting the shRNAs into siRNA-like molecules of about 21-23 nucleotides. (Brummelkamp et al., Science 296:550-553 (2002); Lee et al, (2002). supra; Miyagishi and Taira, Nature Biotechnol. 20:497-500 (2002); Paddison et al. (2002), supra; Paul (2002), supra; Sui (2002) supra; Yu et al. (2002), supra).

siRNAs also include “single-stranded small interfering RNA molecules. “Single-stranded small interfering RNA molecules” (“ss-siRNA molecules” or “ss-siRNA”). ss-siRNA is an active single stranded siRNA molecule that silences the corresponding gene target in a sequence specific manner. Preferably, the ss-siRNA molecule has a length from about 10-50 or more nucleotides. More preferably, the ss-siRNA molecule has a length from about 19-23 nucleotides. In addition to compositions comprising ss-siRNA molecules other embodiments of the invention include methods of making said ss-siRNA molecules and methods (e.g., research and/or therapeutic methods) for using said ss-siRNA molecules.

As used herein, the term “specifically hybridizes” or “specifically detects” refers to the ability of a nucleic acid molecule to hybridize to at least approximately 6 consecutive nucleotides of a sample nucleic acid.

A “target gene” is a gene whose expression is to be selectively inhibited or “silenced,” for example VEGF pathway. This silencing is achieved by cleaving the mRNA of the target gene by an siRNA that is created from an engineered RNA precursor by a cell's RNAi system. One portion or segment of a duplex stem of the RNA precursor is an anti-sense strand that is complementary, e.g., fully complementary, to a section of about 18 to about 40 or more nucleotides of the mRNA of the target gene.

This invention is generally related to treatment and management of angiogenesis by using the VEGF pathway members' genes and their products by inhibiting their expression. One embodiment of this invention is directed to a method comprising contacting the cell with a compound that inhibits the synthesis or expression of one or more of the VEGF, VEGFR, LacCer synthase, PECAM-1 genes in an amount sufficient to cause such inhibition. Without being limited by theory, the inhibition is achieved through selectively targeting VEGF pathway memebers' DNA or mRNA, i.e., by impeding any steps in the replication, transcription, splicing or translation of the genes. The sequence of VEGF, VEGFR, LacCer synthase, PECAM-1, are disclosed in GenBank Accession Nos. AF38663 (GalT-V), AF38664 (GalT-VI), NM_{—}008816, NM_{—}000442, (PECAM1), NM_{—}077435, NM_{—}001025370, NM_{—}001025369, NM_{—}001025368, NM_{—}001025367, NM_{—}003376, NM_{—}001025366, (VEGF), X94263, XM_{—}497921, AB065372, AJ319908, D64016, NM_{—}002019 (VEGFR) and NM_{—}000300, BC005919, (PLA2) which are hereby incorporated by reference in their entirety.

RNAi is a remarkably efficient process whereby double-stranded RNA (dsRNA) induces the sequence-specific degradation of homologous mRNA in animals and plant cells (Hutvagner and Zamore (2002), Curr. Opin. Genet. Dev., 12, 225-232; Sharp (2001), Genes Dev., 15, 485-490). In mammalian cells, RNAi can be triggered by 21-nucleotide (nt) duplexes of small interfering RNA (siRNA) (Chiu et al. (2002), Mol. Cell., 10, 549-561; Elbashir et al. (2001), Nature, 411, 494-498), or by micro-RNAs (miRNA), functional small-hairpin RNA (shRNA), or other dsRNAs which are expressed in-vivo using DNA templates with RNA polymerase III promoters (Zeng et al. (2002), Mol. Cell, 9, 1327-1333; Paddison et al. (2002), Genes Dev., 16, 948-958; Lee et al. (2002), Nature Biotechnol., 20, 500-505; Paul et al. (2002), Nature Biotechnol., 20, 505-508; Tuschl, T. (2002), Nature Biotechnol., 20, 440-448; Yu et al. (2002), Proc. Natl. Acad. Sci. USA, 99(9), 6047-6052; McManus et al. (2002), RNA, 8, 842-850; Sui et al. (2002), Proc. Natl. Acad. Sci. USA, 99(6), 5515-5520.)

The present invention features “small interfering RNA molecules” (“siRNA molecules” or “siRNA”), methods of making said siRNA molecules and methods (e.g., research and/or therapeutic methods) for using said siRNA molecules. A siRNA molecule of the invention is a duplex consisting of a sense strand and complementary antisense strand, the antisense strand having sufficient complementary to a target mRNA to mediate RNAi. Preferably, the strands are aligned such that there are at least 1, 2, or 3 bases at the end of the strands which do not align (i.e., for which no complementary bases occur in the opposing strand) such that an overhang of 1, 2 or 3 residues occurs at one or both ends of the duplex when strands are annealed. Preferably, the siRNA molecule has a length from about 10-50 or more nucleotides, i.e., each strand comprises 10-50 nucleotides (or nucleotide analogs). More preferably, the siRNA molecule has a length from about 16-30, e.g., 16, 17, 18, 19, 20, 21, 22, 23, 24, 25, 26, 27, 28, 29, or 30 nucleotides in each strand, wherein one of the strands is substantially complementary to, e.g., at least 80% (or more, e.g., 85%, 90%, 95%, or 100%) complementary to, e.g., having 3, 2, 1, or 0 mismatched nucleotide(s), a target region, such as a target region that differs by at least one base pair between the wild type and mutant allele, e.g., a target region comprising the gain-of-function mutation, and the other strand is identical or substantially identical to the first strand. small interfering RNA molecules

In one embodiment, the expression of one or more of VEGF, VEGFR, LacCer synthase, PECAM-1 are inhibited by the use of an RNA interference technique referred to as RNAi. RNAi allows for the selective knockdown of the expression of a target gene in a highly effective and specific manner. This technique involves introducing into a cell double-stranded RNA (dsRNA), having a sequence corresponding to the exon portion of the target gene. The dsRNA causes a rapid destruction of the target gene's mRNA. See, e.g., Hammond et al., Nature Rev Gen 2: 110-119 (2001); Sharp, Genes Dev 15: 485-490 (2001), both of which are incorporated herein by reference in their entireties.

Methods and procedures for successful use of RNAi technology are well-known in the art, and have been described in, for example, Waterhouse et al., Proc. Natl. Acad. Sci. USA 95(23): 13959-13964 (1998). The siRNAs of this invention encompass any siRNAs that can modulate the selective degradation of one or more of the VEGF, VEGFR, LacCer synthase, PECAM-1 mRNAs.

The siRNAs of the invention include “double-stranded small interfering RNA molecules” (“ds-siRNA” and “single-stranded small interfering RNA molecules” (“ss-siRNA”), methods of making the siRNA molecules and methods (e.g., research and/or therapeutic methods) for using the siRNA molecules.

Similarly to the ds-siRNA molecules, the ss-siRNA molecule has a length from about 10-50 or more nucleotides. More preferably, the ss-siRNA molecule has a length from about 1545 nucleotides. Even more preferably, the ss-siRNA molecule has a length from about. 1940 nucleotides. The ss-siRNA molecules of the invention further have a sequence that is “sufficiently complementary” to a target mRNA sequence to direct target-specific RNA interference (RNAi), as defined herein, i.e., the ss-siRNA has a sequence sufficient to trigger the destruction of the target mRNA by the RNAi machinery or process. The ss-siRNA molecule can be designed such that every residue is complementary to a residue in the target molecule. Alternatively, substitutions can be made within the molecule to increase stability and/or enhance processing activity of a said molecule. Substitutions can be made within the strand or can be made to residues at the ends of the strand. The 5′-terminus is, most preferably, phosphorylated (i.e., comprises a phosphate, diphosphate, or triphosphate group). The 3′ end of a siRNA may be a hydroxyl group in order to facilitate RNAi, as there is no requirement for a 3′ hydroxyl group when the active agent is a ss-siRNA molecule. Featured are ss-siRNA molecules wherein the 3′ end (i.e., C3 of the 3′ sugar) lacks a hydroxyl group (i.e., ss-siRNA molecules lacking a 3′ hydroxyl or C3 hydroxyl on the 3′ sugar (e.g., ribose or deoxyribose).

The siRNAs of this invention include modifications to their sugar-phosphate backbone or nucleosides. These modifications can be tailored to promote selective genetic inhibition, while avoiding a general panic response reported to be generated by siRNA in some cells. Moreover, modifications can be introduced in the bases to protect siRNAs from the action of one or more endogenous enzymes.

The siRNAs of this invention can be enzymatically produced or totally or partially synthesized. Moreover, the siRNAs of this invention can be synthesized in vivo or in vitro. For siRNAs that are biologically synthesized, an endogenous or a cloned exogenous RNA polymerase may be used for transcription in vivo, and a cloned RNA polymerase can be used in vitro. siRNAs that are chemically or enzymatically synthesized are preferably purified prior to the introduction into the cell.

Although 100 percent sequence identity between the siRNA and the target region is preferred, it is not required to practice this invention. siRNA molecules that contain some degree of modification in the sequence can also be adequately used for the purpose of this invention. Such modifications include, but are not limited to, mutations, deletions or insertions, whether spontaneously occurring or intentionally introduced. Specific examples of siRNAs that can be used to inhibit the expression of one or more of VEGF, VEGFR, LacCer synthase, PECAM-1 are described in detail in Example 7.

The target RNA cleavage reaction guided by siRNAs is highly sequence specific. In general, siRNA containing a nucleotide sequences identical to a portion of the target gene are preferred for inhibition. However, 100% sequence identity between the siRNA and the target gene is not required to practice the present invention. Thus the invention has the advantage of being able to tolerate sequence variations that might be expected due to genetic mutation, strain polymorphism, or evolutionary divergence. For example, siRNA sequences with insertions, deletions, and single point mutations relative to the target sequence have also been found to be effective for inhibition. Alternatively; siRNA sequences with nucleotide analog substitutions or insertions can be effective for inhibition.

Moreover, not all positions of a siRNA contribute equally to target recognition. Mismatches in the center of the siRNA are most critical and essentially abolish target RNA cleavage. In contrast, the 3′ nucleotides of the siRNA do not contribute significantly to specificity of the target recognition. In particular, residue 3′ of the siRNA sequence which is complementary to the target RNA (e.g., the guide sequence) are not critical for target RNA cleavage.

Sequence identity may be determined by sequence comparison and alignment algorithms known in the art. To determine the percent identity of two nucleic acid sequences (or of two amino acid sequences), the sequences are aligned for optimal comparison purposes (e.g., gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, then the molecules are identical at that position. The percent identity between the two sequences is a function of the number of identical positions shared by the sequences (i.e., % homology=# of identical positions/total # of positions×100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (i.e., a local alignment). A preferred, non-limiting example of a local alignment algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10.

In another embodiment, the alignment is optimized by introducing appropriate gaps and percent identity is determined over the length of the aligned sequences (i.e., a gapped alignment). To obtain gapped alignments for comparison purposes, Gapped BLAST can be utilized as described in Altschul et al., (1997) Nucleic Acids Res. 25(17): 3389-3402. In another embodiment, the alignment is optimized by introducing appropriate gaps and percent identity is determined over the entire length of the sequences aligned (i.e., a global alignment). A preferred, non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

Greater than 90% sequence identity, e.g., 91%, 92%, 93%, 94%, 95%, 96%, 97%, 98%, 99% or even 100% sequence identity, between the siRNA and the portion of the target gene is preferred. Alternatively, the ss-siRNA may be defined functionally as a nucleotide sequence (or oligonucleotide sequence) that is capable of hybridizing with a portion of the target gene transcript (e.g., 400 mM NaCl, 40 mM PIPES pH 6.4, 1 mM EDTA, 50 degrees C. or 70 degrees C. hybridization for 12-16 hours; followed by washing). Additional preferred hybridization conditions include hybridization at 70 degrees C. in 1×SSC or 50 degrees C. in 1×SSC, 50% formamide followed by washing at 70 degrees C. in 0.3×SSC or hybridization at 70 degrees C. in 4×SSC or 50 degrees C. in 4×SSC, 50% formamide followed by washing at 67 degrees C. in 1×SSC. The hybridization temperature for hybrids anticipated to be less than 50 base pairs in length should be 5-10 degrees C. less than the melting temperature (Tm) of the hybrid, where Tm is determined according to the following equations. For hybrids less than 18 base pairs in length, Tm(degrees C.)=2(# of A+T bases)+4(# of G+C bases). For hybrids between 18 and 49 base pairs in length, Tm(degrees C.)=81.5+16.6(log 10[Na+])+0.41(% G+C)−(600/N), where N is the number of bases in the hybrid, and [Na+] is the concentration of sodium ions in the hybridization buffer ([Na+] for 1×SSC=0.165 M). Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference. The length of the identical nucleotide sequences may be at least about 10, 12, 15, 17, 20, 22, 25, 27, 30, 32, 35, 37, 40, 42, 45, 47 or 50 bases.

In a preferred aspect, the RNA molecules of the present invention are modified to improve stability in serum or in growth medium for cell cultures. In order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect the efficiency of RNA interference. For example, the absence of a 2′ hydroxyl may significantly enhance the nuclease resistance of the siRNAs in tissue culture medium.

In an embodiment of the present invention the RNA molecule may contain at least one modified nucleotide analogue. The nucleotide analogues may be located at positions where the target-specific activity, e.g., the RNAi mediating activity is not substantially affected, e.g., in a region at the 5′-end and/or the 3′-end of the RNA molecule. Particularly, the ends may be stabilized by incorporating modified nucleotide analogues.

Preferred nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In preferred backbone-modified ribonucleotides the phosphoester group connecting to adjacent ribonucleotides is replaced by a modified group, e.g., of phosphothioate group. In preferred sugar-modified ribonucleotides, the 2′ OH-group is replaced by a group selected from H, OR, R, halo, SH, SR, NH2, NHR, NR2 or ON, wherein R is C₁-C₆ alkyl, alkenyl or alkynyl and halo is F, Cl, Br or I.

Also preferred are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino) propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. It should be noted that the above modifications might be combined.

The nucleic acid compositions of the invention include both siRNA and siRNA derivatives as described herein. For example, cross-linking can be employed to alter the pharmacokinetics of the composition, for example, to increase half-life in the body. Thus, the invention includes siRNA derivatives that include siRNA having two complementary strands of nucleic acid, such that the two strands are crosslinked. The invention also includes siRNA derivatives having a non-nucleic acid moiety conjugated to its 3′ terminus (e.g., a peptide), organic compositions (e.g., a dye), or the like. Modifying siRNA derivatives in this way may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

All documents mentioned herein are incorporated by reference herein in their entirety.

### EXAMPLES

The present invention is further illustrated by the following non-limiting examples.

### Example 1

Reagents—Expanded materials and methods can be found in online data supplement available at http://circres.ahajournals.org

Cell Culture—Human umbilical vein endothelial cells (HUVEC) and endothelial cells growth medium EGM™ were purchased from Cambrex, (Walkersville, Md.) and were cultured in EGM™ medium supplemented with 10% fetal bovine serum (FBS). Human mesoendothelioma cell line [(REN— wild type (WT)] which lacks endogenous PECAM-1 expression and REN (mt-rhPECAM-1) expressing human PECAM-1 was kindly provided by Dr. Steven Albelda, University of Pennsylvania Medical Center, USA. REN-WT was grown in RPMI 1640 supplemented with 101/&FBS. REN (mtrhPECAM-1) was cultured in the same medium with G418 (0.5 g/L Gibco).

Determination of LacCer synthesis—At the indicated time intervals, cells were washed three times with PBS and lipids were extracted and determination of glycosphingolipids by HPTLC was performed as described earlier.¹⁹

Determination of LacCer synthase activity—The activity of LacCer synthase in cells incubated with VEGF was measured employing UDP-[¹⁴C] galactose as a nucleotide sugar donor and glucosylceramide as an acceptor as described earlier.¹⁹

LacCer Synthase (GalT-V siRNA) Synthesis and Transfection—The siRNA sequence for human GalT-V cDNA [Gene Bank Accession No. AF038663] according to the (N19) TT rule was 5′-CGG AGU GAG UGG CUU AAC A dTdT-3′ (sense), 5′-UGU UAA GCC ACU CAC UCC G dTdT-3′ (antisense) respectively. Scrambled (negative control) siRNA used were 5′-AUG GUG AUU AGA CUG UAC C dTdT-3′ (sense), 5′-AAG CGU ACU AGG AUC AGU A dTdT-3^{¢} (antisense), respectively. HUVECs were transfected with siRNA duplexes using Oligofectamine (Invitrogen), following protocol supplied by the manufacturer.

Real-Time Reverse Transcriptase PCR—Real-time RT-PCR was performed with Bio-Rad iCycler system. The primer pairs were designed (Primer Quest—Integrated DNA technologies) and synthesized from 1^{st} BASE (Singapore). The primer sequence for PECAM-1 is as follows: (forward) 5′ TGACCCTTCTGCTCTGTT 3′ and (reverse) 5′ TGAGAGGTGGTGCTGACATC 3′ respectively. For β-actin primers were (forward) 5′ AGGTCATCACTATTGGCAACGA 3′ and (reverse) 5′ CACTTCATGATGGAATTGAATGTAGTT 3′ respectively. Thermal cycling conditions were as follows: initial denaturation at 94° C. for 10 min, followed by 40 cycles of amplification at 94° C./30 s, 60° C./40 s and 72° C./1 min, respectively. Final extension was carried out for 10 min at 72° C.

Western Immunoblot Analysis—25 g of protein was resolved by 10% SDS-PAGE and then transferred to nitrocellulose membrane. The membrane-bound primary antibodies were visualized by horseradish peroxidase-conjugated secondary antibody using chemiluminescence kit. The films were then densitometrically scanned using Molecular Dynamics Image Scanner and analyzed using Image Quant software.

In vitro angiogenesis/tube formation assay—In vitro angiogenesis assay was performed using a commercially available kit from Chemicon Inc (Temecula, Calif.).

Statistical analysis—All assays were performed in duplicate or triplicate and values were expressed as mean±S.D. Student's t test was used to evaluate the statistical significance of data. P<0.05 was considered significant.

### Example 2

### VEGF Induces PECAM-1 mRNA and Protein Expression

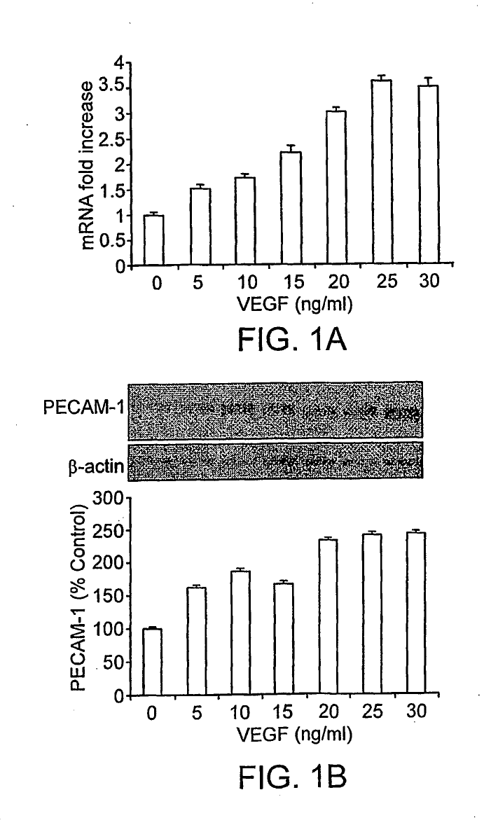

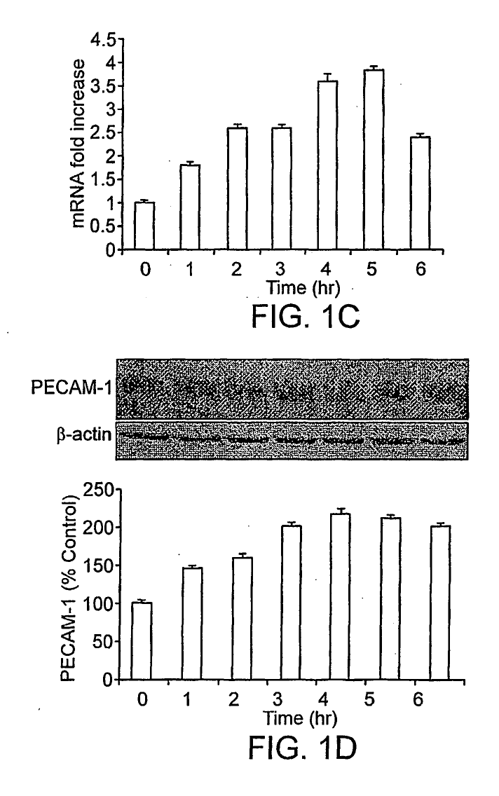

The effect of VEGF on PECAM-1 expression was determined by incubating HUVECs with various concentrations of VEGF (5-30 ng/ml) for different time intervals. There was a dose-dependent increase in the expression of PECAM-1 mRNA. Maximal mRNA expression of PECAM-1 was observed when HUVECs were treated with VEGF (30 ng/ml for 4 hrs) as determined by real-time RT-PCR (FIG. 1A). Similarly, when HUVECs were treated with 25 ng/ml VEGF for different time points, maximal PECAM-1 mRNA expression was observed at 4-5 hrs and decreased thereafter as demonstrated by real-time RT-PCR (FIG. 1B) Further, PECAM-1 protein expression was maximal following incubation with VEGF (30 ng/ml) for 4 hrs (FIG. 1C). When HUVECs were incubated with VEGF (25 ng/ml) for various time intervals, PECAM-1 protein expression was maximal at 4 hrs. (FIG. 1D).

### Example 3

### VEGF Stimulates LacCer Synthesis and this is Abrogated by D-PDMP

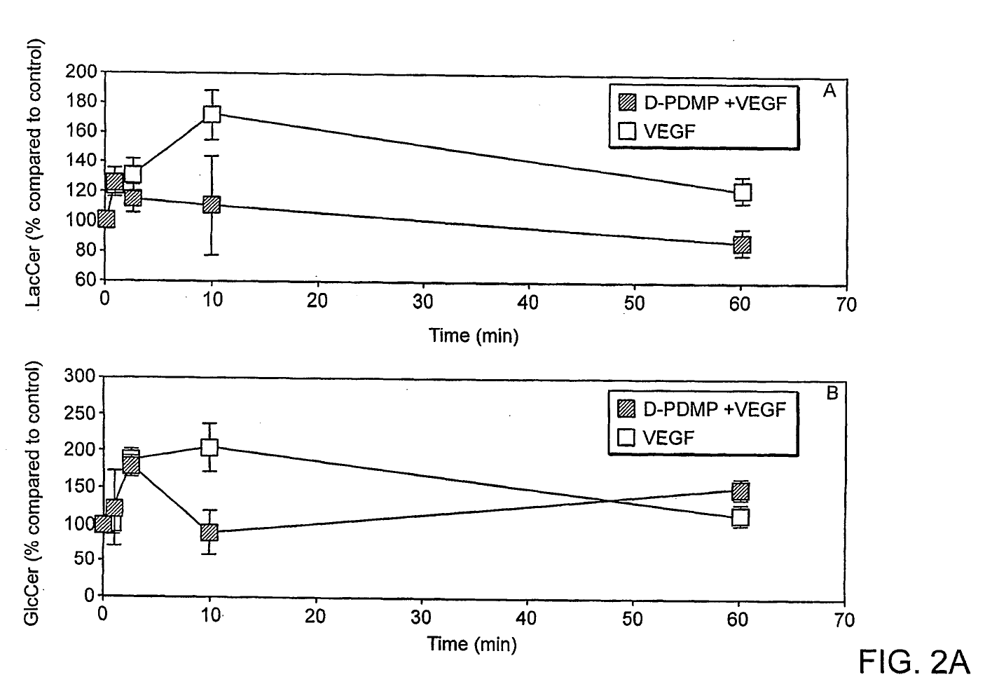

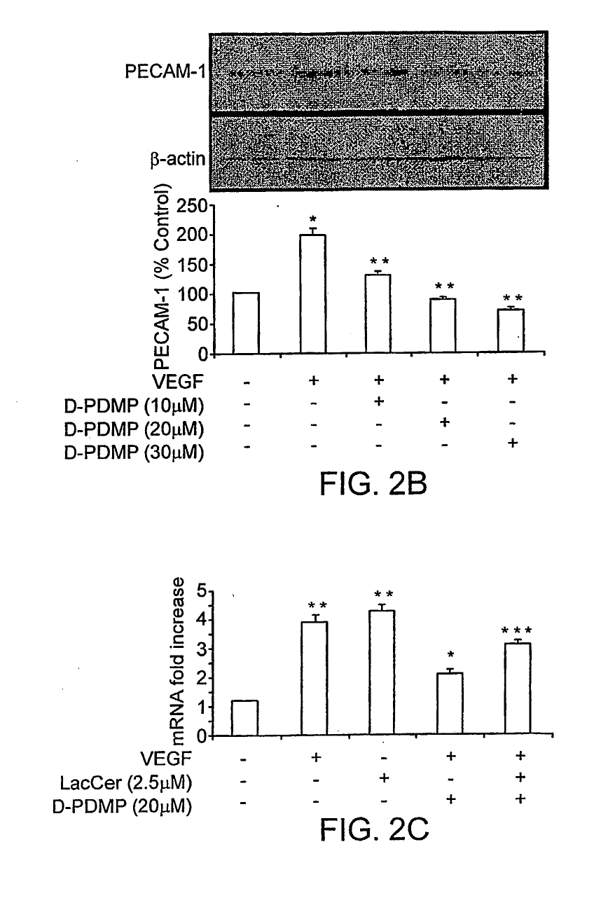

As shown in FIG. 2A, treatment of HUVECs with VEGF (25 ng/ml), significantly stimulated the de novo biosynthesis of LacCer [(FIG. 2A, panel A) open spheres] in a time-dependent fashion, which occurred early at 10 min of incubation but thereafter continued to be higher in VEGF treated cells. In sharp contrast, HUVECs pretreated with 20 μM D-PDMP [(an inhibitor of glycosylceramide synthase; which blocks the synthesis of glucosylceramide (GlcCer) from ceramide) and LacCer synthase], mitigated VEGF induced LacCer biosynthesis [(FIG. 2A, panel A) solid spheres]. VEGF also stimulated the biosynthesis of GlcCer [(FIG. 2A, panel B) open spheres] and D-PDMP pretreatment inhibited VEGF induced GlcCer synthesis as early as 10 min of incubation [(FIG. 2A, panel B, solid squares)]. On the other hand, the level of GbOse3Cer, a product of α-galactosylation of LacCer, synthesis in cells treated with VEGF, with and without D-PDMP was similar (data not shown).

### Example 4

### VEGF Induced PECAM-1 Expression is Abrogated by D-PDMP and Reversed by LacCer

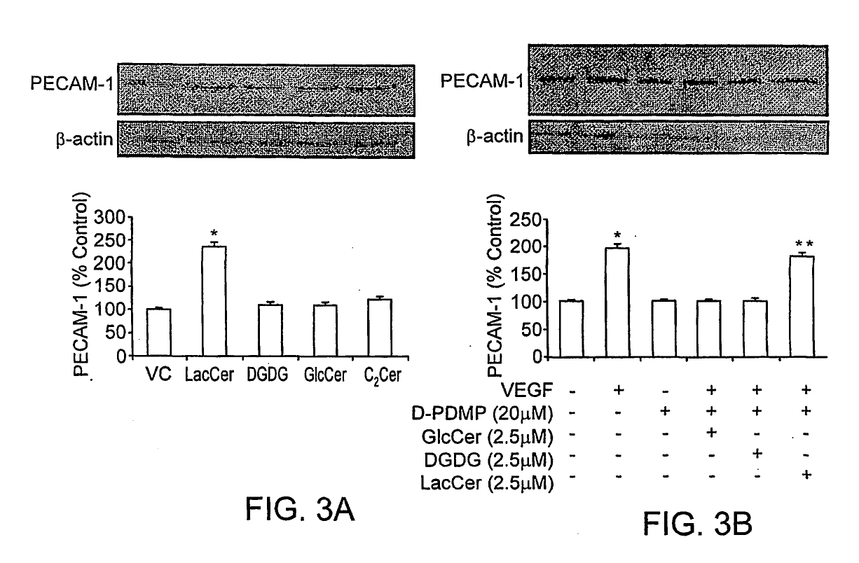

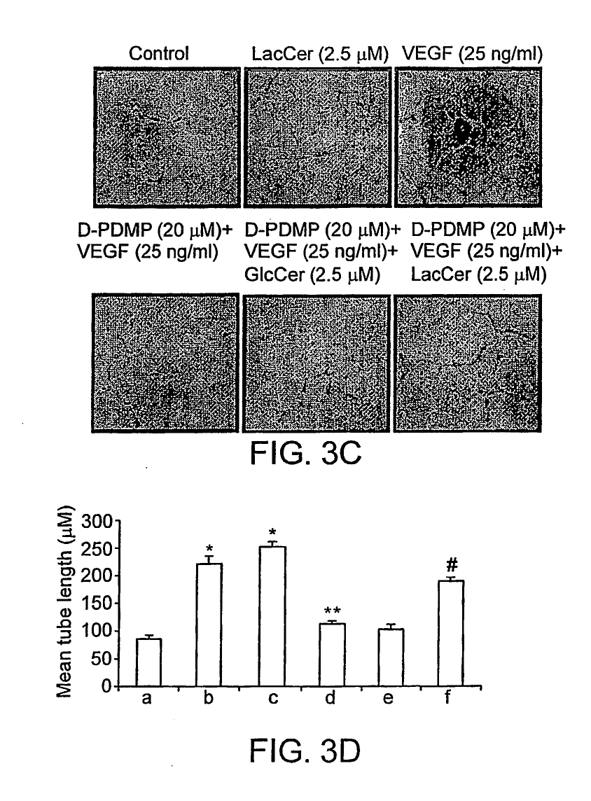

Pre-incubation of HUVECs with D-PDMP (10-30 μM), exerted a concentration-dependent inhibition of VEGF-induced PECAM-1 expression (FIG. 2B). Pre-incubation of HUVECs with D-PDMP (20 μM) for 90 min followed by incubation with VEGF (25 ng/ml) also abrogated PECAM-1 mRNA and protein expression and this was bypassed by LacCer (FIGS. 2C, 3B).

### Example 5

### LacCer Specifically Reversed the Inhibitory Effect of D-PDMP on PECAM-1 Expression and Angiogenesis

When HUVECs were incubated with GlcCer, DGDG or C₂ceramide (2.5 μM each) for 4 hrs, did not induce PECAM-1 expression (FIG. 3A). Moreover, treatment of HUVECs with D-PDMP followed by incubation with GlcCer, DGDG or C2ceramide failed to bypass the inhibitory effect of D-PDMP on VEGF induced PECAM-1 expression (FIG. 3B) and angiogenesis (FIG. 3C—panel e, f and FIG. 3D). In contrast, LacCer significantly induced PECAM-1 expression and angiogenesis independent of the presence/absence of D-PDMP and VEGF (FIGS. 3B, C—panel b and FIG. 3D). These observations suggest that VEGF induced PECAM-1 expression and angiogenesis are tightly associated and regulated by LacCer.

### Example 6

### PPMP Inhibits VEGF Induced PECAM-1 Expression and Angiogenesis and is Bypassed by LacCer

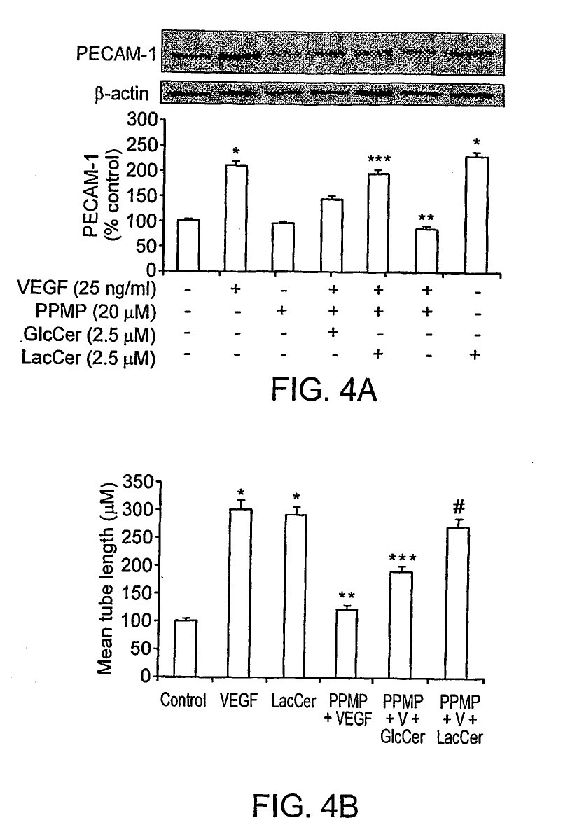

It was found that pretreatment of HUVECs with PPMP (20 μM), a specific inhibitor for glucosylceramide synthase, resulted in mitigation of VEGF induced PECAM-1 expression and angiogenesis (FIGS. 4A, B). LacCer reversed the inhibitory effect of PPMP on VEGF induced PECAM-1 expression and angiogenesis (FIGS. 4A, B). GlcCer also reversed the inhibitory effect of PPMP with regard to PECAM-1 expression and angiogenesis (FIGS. 4A, B) but to a lesser extent as compared to LacCer. Thus these findings suggest that VEGF targeting of LacCer synthase is critical in PECAM-1 expression and angiogenesis in HUVECs.

### Example 7

### LacCer Synthase (GalT-V) Gene Ablation Mitigates PECAM-1 Expression and Angiogenesis

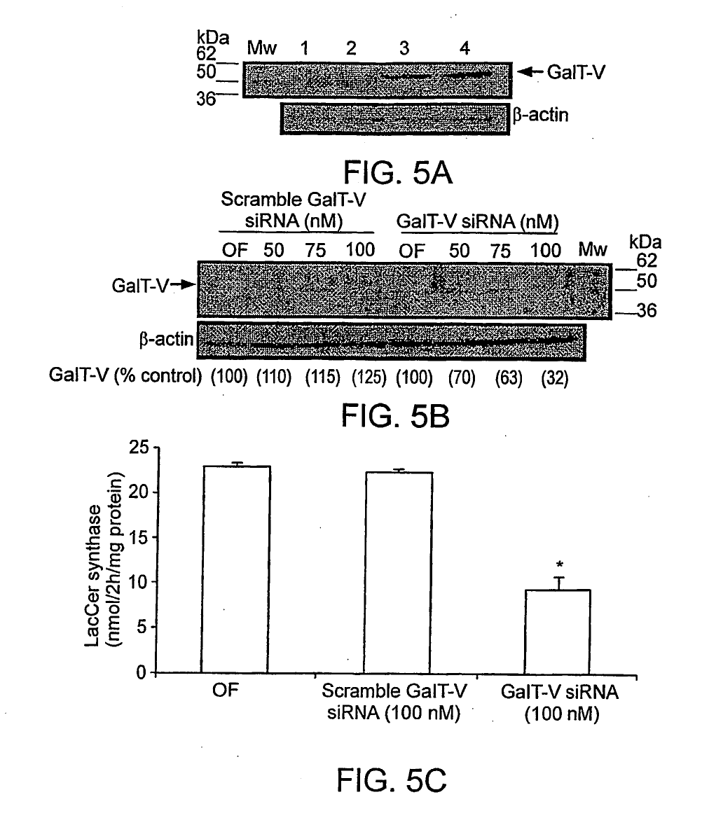

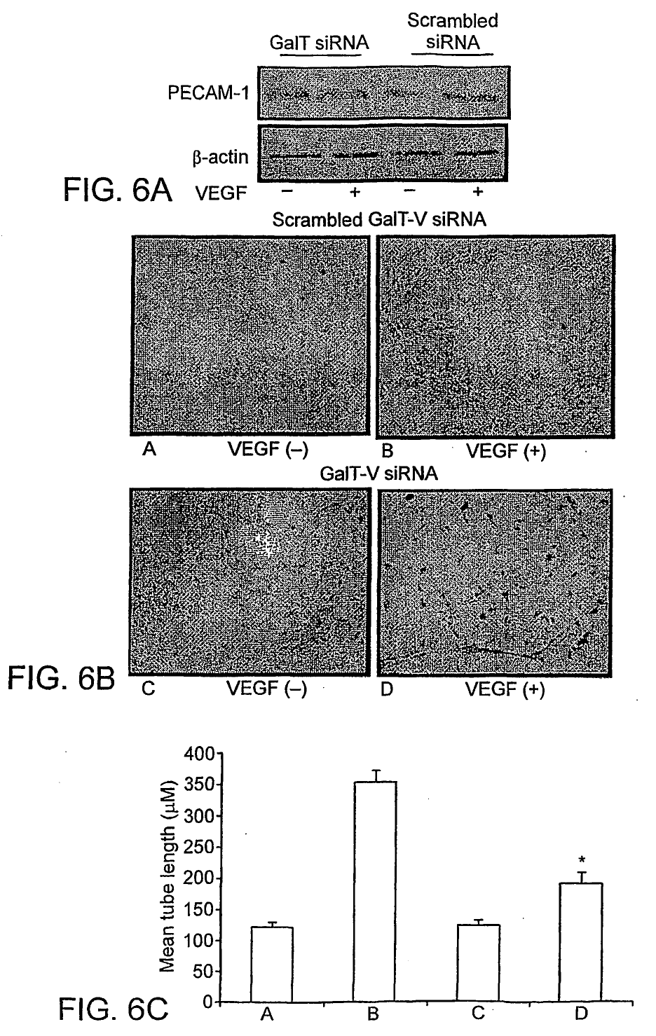

To investigate whether LacCer is specifically required to mediate VEGF induced PECAM-1 expression and angiogenesis we silenced GalT-V gene expression using siRNA duplex directed for human GalT-V. Western immunoblot assay using cell lysates prepared from two separate preparation's of HUVECs and a mutant CHO cell line over expressing GalT-V revealed that the rabbit polyclonal GalT-V antibody (IgG) specifically reacted with GalT-V with an apparent molecular weight of ˜55 kDa (FIG. 5A). Moreover, transfection of GalT-V specific siRNA duplex (100 nM) markedly decreased (˜70% GalT-V) protein expression in HUVEC's (FIG. 5B). Moreover, the activity of GalT-V enzyme in these cells also decreased ˜62% when compared with scrambled siRNA treated cells (FIG. 5C). Further, the effects of VEGF in GalT-V silenced HUVECs on PECAM-1 expression and angiogenesis were investigated. No change was observed in PECAM-1 expression (FIG. 6A) and blunted angiogenesis (FIG. 6 B—panel D and FIG. 6C) in LacCer synthase (GalT-V) silenced cells treated with VEGF when compared to scrambled siRNA transfected cells (FIG. 6B—panel B). These results provide evidence that LacCer is mediates VEGF induced PECAM-1 expression and angiogenesis in HUVECs.

### Example 8

### PECAM-1 is Required for VEGF/LacCer Induced Angiogenesis

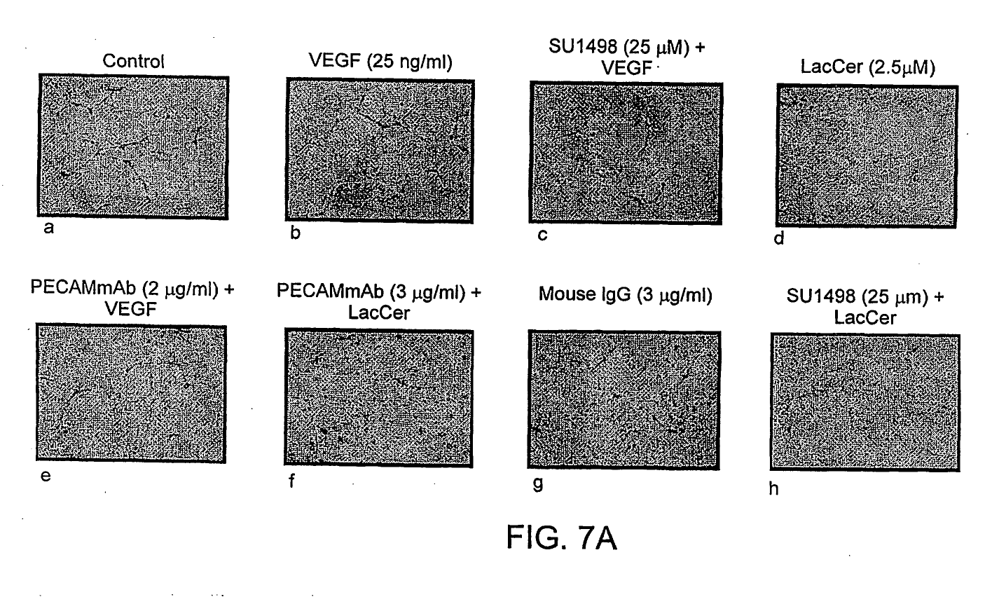

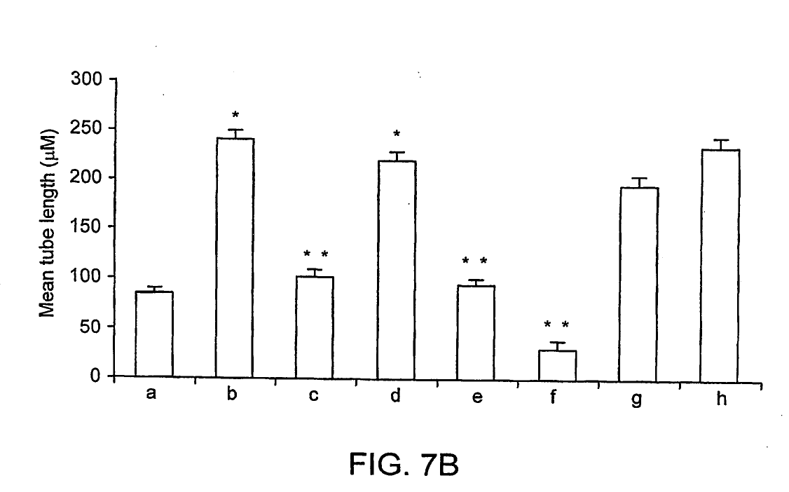

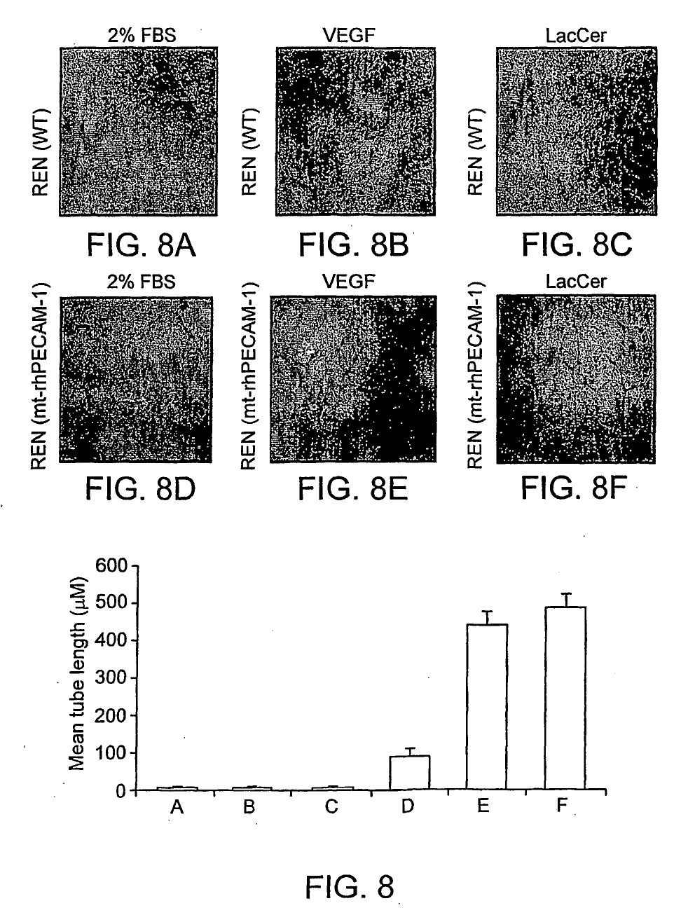

To investigate whether PECAM-1 is absolutely required for VEGF/LacCer induced angiogenesis, we pre-treated HUVECs with PECAM-1 monoclonal antibody, followed by incubation with VEGF or LacCer. In PECAM-1 mAb (monoclonal antibody) but not mouse IgG pre-treated cells, VEGF/LacCer post-treatment did not significantly induce angiogenesis ((FIG. 7A, panels e, f and g). Further, to understand whether PECAM-1 is pivotal for VEGF/LacCer angiogenesis, we performed experiments in REN (WT) cells, which phenotypically resembles endothelial cells, but lacks endogenous PECAM-1 expression. On the other hand, 2.5 kb complete human PECAM-1 cDNA was cloned in to mammalian expression vector pcDNA3 (Invitrogen) under CMV promoter for constitutive expression of PECAM was transfected in REN WT and established REN (mt-rhPECAM-1) as previously described.²⁷ In REN (WT) cells VEGF/LacCer failed to form tube-like structures in the in vitro angiogenesis assays (FIGS. 8 B, C) when compared with 2% FBS treated cells (FIG. 8A). On the other hand, in REN cells transfected with human PECAM-1 gene, VEGF/LacCer induced tube formation in the in vitro angiogenesis assays (FIGS. 8 E,F) when compared with control (FIG. 8D). Therefore, the results from these experiments reveal that PECAM-1 expression is necessary for VEGF/LacCer induced angiogenesis. In addition, it was observed that pre-treatment of HUVECs with VEGF receptor (KDR/Flk-1) antagonist SU 1498 followed by incubation with VEGF but not LacCer failed to induce tube formation/angiogenesis, suggesting the requirement of KDR/Flk-1 for VEGF to elicit angiogenesis (FIGS. 7c,d and h). These observations suggest that, LacCer is downstream of the KDR/Flk-1 in VEGF induced signaling pathway leading to PECAM-1 expression and angiogenesis in HUVEC's.

### Example 9

### PKC and PLA₂ Inhibitors Mitigate LacCer Induced Angiogenesis

PKC inhibitors CC (5.0 μM), GÖ 6850 and 6976 (50 nM) and PLA₂ inhibitors BPB (10 μM) and MAFP (3.0 μM) abrogated VEGF/LacCer induced PECAM-1 expression (See FIG. 1A online data supplement) and tube formation (See FIG. 1B online data supplement) when compared with cells that were treated with vehicle alone (DMSO). This suggests that LacCer induces PECAM-1 expression by recruiting PKC and PLA2 and these are down stream signaling events, which LacCer can recruit to induce PECAM-1 expression and angiogenesis.

### Example 10

### VEGF/LacCer Induce PECAM-1 Expression through NF-kB Activation

Pre-treatment of HUVECs with antioxidants such as NAC, NAPDH oxidase inhibitor (DPI) or NF-κB inhibitor (PDTC), significantly blunted VEGF/LacCer induced PECAM-1 (See FIG. 2A online data supplement) and cytosolic NF-κB expression (See FIG. 2A online data supplement). The results suggest that VEGF induced the de novo synthesis of LacCer, alternatively exogenously added LacCer can trigger free radical generation, presumably via NADP (H) oxidase that could activate NF-kB to induce PECAM-1 expression.¹⁷⁻¹⁹

### Example 11

### L-PDMP Stimulates PECAM-1 Expression and Angiogenesis

It was previously shown that L-PDMP is a potent activator of LacCer synthase^{17,30}. Therefore, it was determined that there is an effect of L-PDMP on PECAM-1 expression and angiogenesis. When cells were treated with increasing concentrations of L-PDMP, it significantly induced PECAM-1 expression (See FIG. 3A online data supplement) and angiogenesis (See FIG. 3B online data supplement). These observations show that PDMP stereoisomers are involved in the up and down regulation of LacCer synthase, PECAM-1 expression and angiogenesis.

To determine the mechanism by which VEGF may recruit LacCer in inducing angiogenesis both pharmacologic and molecular approaches were employed to manipulate enzymes responsible for LacCer biosynthesis. Since VEGF induced LacCer synthase activity, first, we employed D-PDMEP, initially shown to be an inhibitor of GlcCer synthase³³ but later proven to be an inhibitor of purified LacCer synthase.^{30,33,34} Our studies provided evidence that VEGF induced LacCer/GlcCer synthesis, PECAM-1 gene/protein expression and angiogenesis was inhibited by D-PDMP in a dose-dependent fashion. Moreover, this inhibitory effect was by passed by LacCer but not GicCer, suggesting that VEGF targets the LacCer synthase to induce angiogenesis. Recently, Pannu³⁵ demonstrated that IFN (interferon) or lipopolysaccharide (LPS) treatment in neuronal cells also recruited LacCer to induce inducible nitric oxide synthase (iNOS) and accelerated spinal cord injury in mice, TNF-induced proliferation of astrocytes and astrogliosis in spinal cord injury in rats. These events were abrogated by D-PDMP and antisense mediated silencing of LacCer synthase (GalT-2).³⁶

Previously, D-PDMP has also been shown to mitigate neurite out growth and ameliorate osteoclast formation^{37,38} and aortic smooth muscle cell proliferation.¹⁵ Although D-PDMP can also induce apoptosis by raising the cellular level of ceramide, in studies above 37,38 and in the present study D-PDMP (20 μM) up to 4-6 hrs did not induce apoptosis in HUVEC (data not shown). Collectively, D-PDMP has been widely used to elaborate the role of LacCer synthase/LacCer in multiple phenotypic changes in vivo and in vitro. In contrast, a stereoisomer L-PDMP that stimulates the activity of LacCer synthase,³⁰ stimulated PECAM-1 expression and angiogenesis in our present study. Thus stereoisomers of PDMP, by virtue of targeting LacCer synthase, can alter phenotypic changes such as cell proliferation in previous studies¹⁵⁻²² and angiogenesis/tube formation.

To further determine that the target for VEGF action was LacCer synthase and not GlcCer synthase we used PPMP, a specific inhibitor of GlcCer synthase. Again, PPMP like, D-PDMP also mitigated VEGF induced PECAM-1 expression and angiogenesis and this was by passed by LacCer. A more direct approach to ascertain the role of LacCer synthase/LacCer in VEGF induced PECAM-1 expression and angiogenesis in our study was to employ siRNA-mediated gene ablation. The LacCer synthase/GalT-V expression was silenced in HUVEC and then compared its effect on VEGF induced PECAM-1 protein expression and angiogenesis. Results summarized in FIGS. 6A,B suggest that LacCer synthase (GalT-V) siRNA silencing in HUVECs contributed to a ˜70% decrease in the GalT-V gene/protein ablation and mitigated VEGF induced PECAM-1 gene expression and angiogenesis. It was observed that HUVECs also have GalT-VI; another LacCer synthase in addition to GalT-V. However, based upon Northern blot assays, GalT-V constitutes ˜90% of the total LacCer synthase in HUVECs (data not shown).

In our present study REN cells (that are devoid of PECAM-1), were unresponsive to VEGF/LacCer induced angiogenesis. In contrast, VEGF/LacCer treatment in REN cells expressing full-length cDNA for PECAM-1 responded strongly in regard to PECAM-1 expression and formation of tube like structures in the in vitro assay of angiogenesis (FIG. 8). It was also observed that the use of PECAM-1 antibody in HUVECs mitigated angiogenesis^{10,11}. Thus, both pharmacological and/or genetic manipulations of LacCer synthase adversely affected PECAM-1 gene/protein expression and angiogenesis.

Using specific inhibitors of PKC/PLA₂, in the present study it was found that VEGF induced de novo synthesis of LacCer, that in turn recruits PKC/PLA₂ to induce angiogenesis/tube formation in HUVECs. In addition, we have also documented that in a pro-monocytic cell line (U-937), LacCer specifically stimulated the translocation of cytosolic PKC α/ε to the cell membrane considered to be due to the activation of these proteins.²²

Recently, the requirement for sphingosine-1-phosphate receptor-1 in tumor angiogenesis was demonstrated using in vivo RNA interference.⁴¹ Present study points to the direction that since angiogenesis is a critical multifaceted physiologic event, cells may recruit various sphingolipids to meet the demands of organ repair, growth and development. Our present study indicates that the use of antibodies against PECAM-1, LacCer synthase inhibitors and/or GalT-V siRNA can mitigate VEGF induced angiogenesis and well may serve as invaluable pharmacological reagents in anti-angiogenic therapy.

The invention has been described in detail with reference to preferred embodiments thereof. However, it will be appreciated that those skilled in the art, upon consideration of this disclosure, may make modification and improvements within the spirit and scope of the invention as set forth in the following claims.

### REFERENCES

1. Folkman J C. Angiogenesis in cancer, vascular, rheumatoid and other diseases. Nat. Med. 1995; 1: 27-31.
2. Neufeld G, Cohen T, Gengrinovitch S, Poltrorak Z. Vascular endothelial growth factor (VEGF) and its receptors. FASEB J 1999; 13: 9-22
3. Ferrara N. Vascular endothelial growth factor. Eur J Cancer. 1996; 32: 2413-2422.
4. Petrova T V, Makinen T, Alitalo K VEGF expression in human coronary atherosclerotic lesions: possible pathophysiological significance of VEGF in progression of atherosclerosis. Exp Cell Res. 1990; 253: 117-130.
5. Brasen J H, Kiuelä A, Röser K, Rissanen T T, Niemi M, Luft F C, Donath K, YlaHerttualas. Angiogenesis, VEGF and platelet-derived growth factor BB expression, iron deposition and oxidation-specific epitopes in stented human coronary arteries. Arterioscler Throm Vas Biol. 2001; 21: 1720-1726.
6. Zhao Q, Egashira K, Inoue S. Vascular endothelial growth factor is necessary in the development of atherosclerosis by recruiting monocytes in a rat model of long-term inhibition of nitric oxide synthesis. Circulation. 2002; 105: 1110-1115.
7. Newman P J. The biology of PECAM-1. J Clin Invest. 1997; 99: 3-8.
8. Ilan N, Madri J A. PECAM-1: old friend, new partners. Cuir Opin Cell Biol. 2003; 15: 515-524.
9. DeLisser H M, Solomindu C M, Strieter R M. Involvement of endothelial PECAM-1/CD31 in angiogenesis. Am J Pathol. 1997; 151: 671-677
10. Cao G, Obrein C D, Zhou Z, Sanders S M, Greenbaum N J, DeLisser H M. Involvement of human PECAM-1 in angiogenesis and in vitro endothelial cell migration. Am J Cell Physiol. 2002; 282: C1181-1190.
11. Ji G, O'Brien C D, Feldman M, Manevich Y, Lim P, Sun J, Albelda S M, Kotlikoff M I. PECAM-1 (CD31) regulates a hydrogen peroxide-activated nonselective cation channel in endothelial cells. J. Cell Biol. 2002; 157:173-184.
12. Zhou Z, Christofidou-Solomildou M, Garlanda C, DeLisser H M. Antibody against murine PECAM-1 inhibits tumor angiogenesis in mice. Angiogenesis. 1999; 3:181-188.
13. O'Brein C D, Cao G, Makriginnakis A, DeLisser H M. Role of immunoreceptor tyrosine-based motifs of PECAM-1 in PECAM-1 dependent cell migration. Am J Physiol Cell Physiol. 2004; 287: C1103-1113.
14. Hakomori S I, Igarashi Y. Gangliosides and glycosphingolipids as modulators of cell growth, adhesion, and transmembrane signaling. Adv. Lipid Res. 1993; 25: 147-162
15. Chatterjee S. Lactosylceramide stimulates aortic smooth muscle cell proliferation. Biochem Biophys Res Commun. 1991; 181:554-561.
16. Chatterjee, S., Bhunia, A. K.; Snowden, A.; Han, H. Oxidized low density lipoproteins stimulate galactosyltransferase activity, ras activation, p44 mitogen activated protein kinase and c-fos expression in aortic smooth muscle cells. Glycobiology 1997; 7:703-710
17. Bhunia A K, Han H, Snowden A, Chatterjee S. Redox-regulated signaling by lactosylceramide in the proliferation of human aortic smooth muscle cells. J Biol Chem. 1997; 271:15642-15649.
18. Chatterjee S. Oxidized LDL and lactosylceramide both stimulate the expression of proliferating cell nuclear antigen and proliferation of aortic smooth muscle cells. Indian J Biochem Biophys. 1997; 34: 56-61
19. Bhunia A K, Arai T, Bulkey G, Chatterjee S. Lactosylceramide mediates tumor necrosis factor- (TNF-)induced intercellular adhesion molecule-1 (ICAM-1) expression and the adhesion of neutrophil in human umbilical vein endothelial cells. J Biol. Chem. 1998; 273: 34349-34357.
20. Arai T, Bhunia A K, Chatterjee S, Bulkley G B. Lactosylceramide stimulates human neutrophils to upregulate Mac-1, adhere to endothelium, and generate reactive oxygen metabolites in vitro. Circ. Res. 1998; 82: 540-547.
21. Chatterjee S, Shi W Y, Wilson P, Mazumder A. Role of lactosylceramide and MAP kinase in the proliferation of proximal tubular cells in human polycystic kidney disease. J Lipid Res. 1996; 37: 1334-1344.
22. Gong N, Wei H, Chowdhury H S, Chatterjee S. Lactosylceramide recruits PKC α/ε and phospholipase A2 to stimulate PECAM-1 expression and adhesion of human monocytes to endothelial cells. (2004) Proc Natl Acad Sci U.S.A. 2004; 101:
23. Chatterjee S, Sekerke C S, Kwiterovich P O. Increased accumulation of glycosphingolipids in familial hypercholesterolemia. J Lipid Res. 1982; 23: 513522.
24. Mukhin D, Chao F F, Kruth H S. Glycosphingolipid accumulation in the aortic wall is another feature of human atherosclerosis. Arterioscler Thromb Vasc Biol. 1995; 15: 1607-1615.
25. Chatterjee S B, Dey S, Shi W Y, Thomas K, Hutchins G M. Accumulation of glycosphingolipids in human atherosclerotic plaque and unaffected aorta tissues. Glycobiology 1997; 7: 57-65.
26. Serebruany V L, Murugesan S R, Pothula A, Semaan H, Gurbel P A. Soluble PECAM1, but not P-selectin, nor osteonectin identify acute myocardial infarction in patients presenting with chest pain. Cardiology 1999; 91: 50-55.
27. Zibara K, Chignier E, Covacho C, Poston R, Canard G, McGregor J. Modulation of expression of endothelial intercellular adhesion molecule-1 (ICAM-1), PECAM-1 and VCAM-1 in aortic arch lesions in apolipoprotein E-deficient compared with wild-type mice. Arterioscler Thromb Vasc Biol. 2000; 20: 2288-2296.
28. Indira Gurubhagavatula, Yassine Amrani, Domenico Pratico, Frederick L. Ruberg, Steven M. Albelda, and Reynold A. Panettieri, Jr. Engagement of human PECAM-1 (CD 31) on human endothelial cells increases intracellular calcium ion concentration and stimulates prostacyclin release. J Clin Invest. 1998; 101: 212-222.
29. Iwabuchi K, Nagaoka I. Lactosylceramide-enriched glycosphingolipid signaling domain mediates superoxide generation in human neutrophils. Blood. 2002; 24:59425954.
30. Chatterjee S, Cleveland T, Shi W Y, Inokuchi J, Radin N S. Studies of the action of ceramide-like substances (D- and L-PDMP) on sphingolipid glycosyltransferases and purified lactosylceramide synthase. Glycoconj J. 1996; 13: 481-486.
31. Dvorak H F, Nagy J F, Feng D, Brown L F, Dvorak A M. Vascular permeability factor/VEGF and the significance of microvascular hyperpermiability in angiogenesis. Curr Top Microbiol Immunuol. 1999; 237: 97-132.
32. Strawn L M, McMahon G, App H, Schreck R, Kuchler W, Longhi M P, Hui H, Tang C, Levitzld A, Gazit A, Chen I et al. FLK-1 as a target for tumor inhibition. Cancer Res. 1996; 56: 3540-3545.
33. Inokuchi J, Radin N S. Preparation of the active isomer of 1-phenyl-2-decanoylamino-3-morpholino-1-propanol, inhibitor of murine glucocerebroside synthase. J Lipid Res. 1987; 28: 565-571.
34. Chatterjee S, Ghosh N. Oxidized low density lipoprotein stimulates aortic smooth muscle cell proliferation. Glycobiology 1996; 6:303-311
35. Pannu S, Won J S, Khan M, Singh A K, Singh I. A novel role of lactosylceramide in the regulation of lipopolysaccharide/interferon-g mediated iNOS synthase gene expression: implications for neuroinflammatory diseases. J Neurosci. 2004; 24: 5942-5954.
36. Pannu R, Singh A K, Singh I. A novel role of lactosylceramide in the regulation of TNF-α-mediated proliferation of rat primary astrocytes. Implications for astrogliosis following neurotrauma. J Biol. Chem. 2005; 280: 316-324.
37. Mutoh T, Tokuda A, Inokuchi J, Kuriyama M. Glucosylceramide sythase inhibitor inhibits the action of nerve growth factor in PC 12 cells. J Biol. Chem. 1998; 273: 26001-26007.
38. Iwamoto T, Fukumoto S, Kanaoka K, Sakai E, Shibata M, Furukawa K, Kato Y, Mizano A. Lactosylceramide is essential for osteoclastogenesis mediated by macrophage-colony-stimulating factor and receptor activator of nuclear factor kB ligand. J Biol Chem. 2001; 276: 46031-46038.
39. Gliki G, Wheeler-Jones C, Zachary I. Vascular endothelial growth factor induces protein kinase C (PKC)-dependent Akt/PKB activation and phosphatidylinositol 3′ kinase-mediates PKC delta phosphorylation: role of PKC in angiogenesis. Cell Biol Int. 2002; 26:751-759.
40. Xia P, Aiello L P, Ishii H, Jiang Z Y, Park D J, Robinson G S, Takagi H, Newsome W P, Jirousek M R, King G L. Characterization of vascular endothelial growth factor's effect on the activation of protein kinase C, its isoforms, and endothelial cell growth. J Clin Invest. 1996; 98:2018-2026.
41. Chae S, Paike J H, Fumeaux H, Hla T. Requirement of sphingosine 1-phosphate receptor-1 in tumor angiogenesis demonstrated by in vivo RNA interference. J Clin Invest. 2004; 114: 1082-1089.

What is claimed is:

1. A method for treating a subject suffering from or susceptible to a disease or condition involving angiogenesis comprising administering to the subject a therapeutically effective amount of a vascular endothelial growth factor (VEGF) pathway inhibitor.

2. The method of claim 1 wherein the disease or condition involving angiogenesis is cancer, coronary heart disease, tumor metastasis, inflammatory vascular disease, or diabetes.

3. The method of claim 1, wherein the VEGF pathway comprises the interaction or involvement of one or more of lactosylceramide synthase (LacCer synthase), VEGF, vascular endothelial growth factor receptor (VEGFR), platelet endothelial cell adhesion molecule 1 (PECAM-1), lactosylceramide (LacCer), or PLA2.

4. The method of claim 1, wherein the VEGF pathway inhibitor is a compound of Formula I: 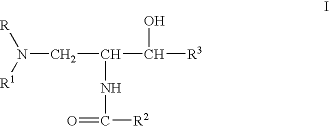
wherein R and R¹ are independently selected from the group consisting of hydrogen and straight-chained or branched C₁-C₆ alkyl with or without a substituent, and further wherein R and R¹ may be joined to form a 5, 6 or 7-membered ring;
R² is selected from the group consisting of branched or straight-chained C₆-C₃₀ alkyl with or without one to three double bonds; and
R³ is selected from the group consisting of straight-chained or branched C₈-C₂₀ alkyl with or without one to three double bonds and aryl or substituted aryl where the substituent is halo, C₁-C₄ alkoxy, methylenedioxy, C₁-C₄ mercapto, amino or substituted amino in which the amino substituent may be C₁-C₄ alkyl, or a pharmaceutically acceptable salt thereof.

5. The method of claim 4 wherein R and R¹ are joined to form a 5, 6 or 7-membered ring.

6. The method of claim 5, wherein R and R¹ are joined to form a pyrrolidino, morpholino, thiomorpholino, piperidino or azacycloheptyl ring.

7. The method of claim 1, wherein the VEGF pathway inhibitor is one or more of 1-phenyl-2-decanoylamino-3-morpholino-1-propanol;
1-phenyl-2-hexadecanoylamino-3-morpholino-1-propanol;
1-phenyl-2-hexadecanoylamino-3-piperidino-1-propanol;
1-phenyl-2-hexadecanoylamino-3-pyrrolidino-1-propanol;
1-morpholino-2-hexadecanoylamino-3-hydroxyoctadec-4,5-ene;
1-pyrrolidino-2-hexadecanoylamino-3-hydroxyoctadec-4,5-ene; (1R,2R)-1-phenyl-2-decanoylamino-3-morpholino-1-propanol (D-PDMP); or trans-(2R,3R)-1-pyrrolidino-2-hexadecanoylamino-3-hydroxyoctadec-4,5-ene, chelerythrine chloride.

8. The method of claim 1, wherein the VEGF pathway inhibitor is one or more of SU-1498, Gö6976, Gö6850, bromophenacyl bromide (BMB), methyl-arachidonyl fluorophosphonate (MAFP), pyrrolidine carbodithioicacid, diphenylene iodonium chloride and N-acetyl-L-cysteine; PECAM-1, PLA2, LacCer or LacCer synthase antibodies or fragments thereof; PECAM-1, PLA2, LacCer or LacCer synthase peptides;
or PECAM-1, PLA2, LacCer or LacCer synthase RNAi.

9. The method of claim 8, wherein the RNAi is one or more of 5′-CGG AGU GAG UGG CUU AAC A dTdT-3′ (SEQ ID NO: 17) (sense), 5, UGU UAA GCC ACU CAC UCC G dTdT-3′ (SEQ ID NO: 18) (antisense) or fragments or variants thereof.

10. The method of claim 8, wherein the PECAM-1, PLA2, LacCer or LacCer synthase antibody is specific for
IGAQVYEQVLRSAYAKRNSSVND (SEQ ID NO: 1) (GalT-V); IGMHM-----RLYTNKNSTLNGT (SEQ ID NO: 2) (GalT-VI);
VLENSTKNSNDPAVFKDNPTEDVEYQCVADN (SEQ ID NO: 26) (PECAM-1); PERLP (SEQ ID NO: 3); PTIKLGGHWKP (SEQ ID NO: 4); PRWKVAILIP (SEQ ID NO: 5);
PFRNRHEHLP (SEQ ID NO: 6); PVLFRHLLP (SEQ ID NO: 7); PEGDTGKYKSIP (SEQ ID NO: 8); PENFTYSP (SEQ ID NO: 9); PYLP (SEQ ID NO: 10); PCPEKLP (SEQ ID NO: 11);
PGGHWRP (SEQ ID NO: 12); PRWKVAVLIP (SEQ ID NO: 13); PFRNRHEHLP (SEQ ID NO: 6); PIFFLHLIP (SEQ ID NO: 14); PEGDLGKYKSIP (SEQ ID NO: 15); PELAP (SEQ ID NO: 16); CC(P)-x-H-(LGY)-x-C (SEQ ID NO: 19), wherein histidine H is the active site of the enzyme (PLA2), or fragments or variants thereof.

11. The method of claim 8, wherein the PECAM-1, PLA2, LacCer or LacCer synthase peptide is one or more of
IGAQVYEQVLRSAYAKRNSSVND (SEQ ID NO: 1) (GalT-V); IGMHMI-----RLYTNKNSTLNGT (SEQ ID NO: 2) (GalT-VI);
VLENSTKNSNDPAVFKDNPTEDVEYQCVADN (SEQ ID NO: 26) (PECAM-1); PERLP (SEQ ID NO: 3); PTIKLGGHWKP (SEQ ID NO: 4); PRWKVAILIP (SEQ ID NO: 5);
PFRNRHEHLP (SEQ ID NO: 6); PVLFRHLLP (SEQ ID NO: 7); PEGDTGKYKSIP (SEQ ID NO: 8); PENFTYSP (SEQ ID NO: 9); PYLP (SEQ ID NO: 10); PCPEKLP (SEQ ID NO: 11);
PGGHWRP (SEQ ID NO: 12); PRWKVAVLIP (SEQ ID NO: 13); PFRNRHEHLP (SEQ ID NO: 6); PIFFLHLIP (SEQ ID NO: 14); PEGDLGKYKSIP (SEQ ID NO: 15); PELAP (SEQ ID NO: 16); CC(P)-x-H-(LGY)-x-C (SEQ ID NO: 19), wherein histidine H is the active site of the enzyme, or fragments or variants thereof.

12. The method of claim 1, further comprising identifying the subject as in need of treatment for a disease or condition involving angiogenesis.

13. The method of claim 12, wherein the identification compromises diagnosis of cancer, coronary heart disease, tumor metastasis, inflammatory vascular disease, ischemia-reperfusion injury, hypertension, or diabetes.

14. The method of claim 1 wherein a VEGF pathway inhibitor is administered to the subject orally, intramuscularly, intratumorally, stent, or intraperitoneally.

15. The method of claim 1, wherein a therapeutically effective amount of a VEGF inhibitor mitigates VEGF-induced in vitro angiogenesis/tube formation.

16. The method of claim 1, wherein the VEGF pathway inhibitor is one or more of coated on or contained within a medical device.

17. The method of claim 16, wherein the medical device comprises a biodegradable biopolymer.

18. The method of claim 17, wherein the medical device is a stent.

19. A method for determining the therapeutic capacity of a VEGF pathway inhibitor to reduce angiogenesis in a subject, comprising:
performing an invasive surgical procedure on the subject;
administering a VEGF pathway inhibitor to the subject; and
examining the subject for vessel growth.

20. The method of claim 19, wherein the invasive surgical procedure is a tumor removal.

21. The method of claim 19, wherein the subject is a animal model.

22. The method of claim 21, wherein the animal model is a tumor xenograft.

23. A method for determining the therapeutic capacity of a VEGF pathway inhibitor to reduce angiogenesis in a subject, comprising:
determining pre-treatment levels of angiogenesis in a subject;
administering a therapeutically effective amount of a VEGF pathway inhibitor to the subject; and
determining a post-treatment level of angiogenesis in the subject.

24. The method of claim 23, wherein a decrease in the angiogenesis indicated that the VEGF pathway inhibitor is efficacious.

25. The method of claim 23, wherein the pre-treatment and post-treatment levels of angiogenesis are determined in a diseased tissue.

26. The method of claim 23, wherein the diseased tissue is one or more of lung, heart, liver, tumor, or vasculature.

27. The method of claim 23, wherein the level of angiogenesis is determined by PECAM-1 expression, GatT-V expression, tube formation, or LacCer level.

28. A method for determining the therapeutic capacity of a candidate VEGF pathway inhibitor for treating angiogenesis, comprising:
providing a population of cells;
contacting the cells with a candidate composition, and
determining effect of the candidate composition on one or more of PECAM-1 expression, GatT-V expression, tube formation, or LacCer level.

29. The method of claim 28, further comprising contacting the cells with VEGF prior to contacting the cells with the candidate compound.

30. The method of claim 28, further comprising contacting the cells with VEGF after the contacting the cells with the candidate compound.

31. A method for treating a subject suffering from or susceptible to tissue degeneration comprising administering to the subject a therapeutically effective amount of a vascular endothelial growth factor (VEGF) pathway activator.

32. The method of claim 31, wherein the tissue degeneration is related to intrauterine growth of a fetus, systemic sclerosis, wound healing, ischemia, reperfusion injury, diabetes, coronary artery disease, tumor growth.

33. The method of claim 31, wherein the VEGF pathway comprises the interaction or involvement of one or more of lactosylceramide synthase (LacCer synthase), VEGF, vascular endothelial growth factor receptor (VEGFR), platelet endothelial cell adhesion molecule 1 (PECAM-1), lactosylceramide (LacCer), or PLA2.

34. The method of claim 31, wherein the VEGF pathway activator is an L isomer a compound of Formula I: 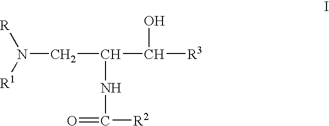
wherein R and R¹ are independently selected from the group consisting of hydrogen and straight-chained or branched C₁-C₆ alkyl with or without a substituent, and further wherein R and R¹ may be joined to form a 5, 6 or 7-membered ring;
R² is selected from the group consisting of branched or straight-chained C₆-C₃₀ alkyl with or without one to three double bonds; and
R³ is selected from the group consisting of straight-chained or branched C₆-C₂₀ alkyl with or without one to three double bonds and aryl or substituted aryl where the substituent is halo, C₁-C₄ alkoxy, methylenedioxy, C₁-C₄ mercapto, amino or substituted amino in which the amino substituent may be C₁-C₄ alkyl, or a pharmaceutically acceptable salt thereof.

35. The method of claim 34, wherein R and R¹ are joined to form a 5, 6 or 7-membered ring.

36. The method of claim 35, wherein R and R¹ are joined to form a pyrrolidino, morpholino, thiomorpholino, piperidino or azacycloheptyl ring.

37. The method of claim 31, wherein the VEGF pathway inhibitor is one or more of the L isomer of 1-phenyl-2-decanoylamino-3-morpholino-1-propanol;
1-phenyl-2-hexadecanoylamino-3-morpholino-1-propanol;
1-phenyl-2-hexadecanoylamino-3-piperidino-1-propanol;
1-phenyl-2-hexadecanoylamino-3-pyrrolidino-1-propanol;
1-morpholino-2-hexadecanoylamino-3-hydroxyoctadec-4,5-ene;
1-pyrrolidino-2-hexadecanoylamino-3-hydroxyoctadec-4,5-ene.

38. The method of claim 31, further comprising identifying the subject as in need of treatment for tissue degeneration.

39. The method of claim 38, wherein a VEGF pathway activator is administered to the subject orally, intramuscularly, intra-tumorally, stent, or intraperitoneally.

40. A method for determining the therapeutic capacity of a VEGF pathway activator to reduce tissue degeneration in a subject, comprising:
determining pre-treatment levels of tissue degeneration in a subject;
administering a therapeutically effective amount of a VEGF pathway activator to the subject; and
determining a post-treatment level of tissue degeneration in the subject.

41. The method of claim 40, wherein a decrease in the tissue degeneration indicates that the VEGF pathway activator is efficacious.

42. The method of claim 40, wherein the pre-treatment and post-treatment levels of tissue degeneration are determined in a diseased tissue.

43. The method of claim 40, wherein the diseased tissue is one or more of a fetus, lung, heart, liver, vasculature or nervous tissue.

44. The method of claim 43, wherein vasculature is one or more of cardiac ventricular microvessel formation to increase collateral blood flow to the heart or other tissue.

45. The method of claim 40, wherein the level of tissue degeneration is determined by PECAM-1 expression, GalT-V expression, tube formation, or LacCer level.

46. A method for determining the therapeutic capacity of a candidate VEGF pathway activator for treating tissue degeneration, comprising:
providing a population of cells;
contacting the cells with a candidate composition, and
determining effect of the candidate composition on one or more of PECAM-1 expression, GalT-V expression, tube formation, or LacCer level, wherein an increase in one or more of PECAM-1 expression, GalT-V expression, tube formation, or LacCer level indicates that the candidate composition may be efficacious.

47. A method for treating a subject suffering from or susceptible to a disease or condition involving angiogenesis comprising administering to the subject a therapeutically effective amount of a vascular endothelial growth factor (VEGF) pathway inhibitor and a VEGF pathway activator.

48. The method of claim 47, wherein the inhibitor mitigates angiogenesis in certain tissues and the activator promotes growth in others.

49. The method of claim 47, wherein the inhibitor and activator are coated on or are within a biodegradable biopolymer.

50. The method of claim 47, wherein the inhibitor and activator are coated on nano-particles.
